(19) United States
(12) Patent Application Publication
(10) Pub. No.: US 2025/0161207 A1
(43) Pub. Date: May 22, 2025

(54) ORAL PRODUCTS, THREE-DIMENSIONAL PRINTERS USED FOR FORMING ORAL PRODUCTS, AND THREE-DIMENSIONAL PRINTING METHODS FOR FORMING ORAL PRODUCTS

(71) Applicant: Altria Client Services LLC, Richmond, VA (US)

(72) Inventors: Eve MARIN, Whitsett, NC (US); Kevin ROMAN, Marietta, GA (US); Izabela SLIWINSKA, Marietta, GA (US)

(73) Assignee: Altria Client Services LLC, Richmond, VA (US)

(21) Appl. No.: 18/953,835

(22) Filed: Nov. 20, 2024

Related U.S. Application Data
(60) Provisional application No. 63/600,789, filed on Nov. 20, 2023.

(51) Int. Cl. A61K 9/00; A61K 31/465; B33Y 10/00; B33Y 40/10; B33Y 80/00

(52) U.S. Cl. CPC A61K 9/0056; A61K 31/465; B33Y 10/00; B33Y 40/10; B33Y 80/00

(57) ABSTRACT

A method of making an oral product includes printing a first product portion of an oral product using of a three-dimensional printer. The first product portion has a first composition. The method further includes printing a second product portion of the oral product using the three-dimensional printer, the second product portion having a second composition different from the first composition, the first product portion and the second product portion defining distinct, non-mixed regions of the oral product.

### CROSS-REFERENCE TO RELATED APPLICATION

This application claims the benefit of U.S. Provisional Application No. 63/600,789, filed on Nov. 20, 2023, the disclosure of which is incorporated herein by reference thereto in its entirety.

### BACKGROUND

### Field

The present disclosure relates to oral products, three-dimensional printers used for forming the same, and three-dimensional printing methods of forming the same.

### Description of Related Art

Oral nicotine products are available in a variety of formats, including, for example, gums, sprays, lozenges, dissolvable tablets, non-dissolvable chews, films, gels, capsules, sticks (for example, coated wooden dowels and/or singular dissolvable sticks), and pouches (for example, containing fivers or granules). Oral products may have selected nicotine levels to create familiar experiences for adult tobacco consumers.

### SUMMARY

At least one example embodiment relates to a method of making an oral product.

In at least one example embodiment, the method includes printing a first product portion of an oral product using of a three-dimensional printer. The first product portion has a first composition. The method further includes printing a second product portion of the oral product using the three-dimensional printer, the second product portion having a second composition different from the first composition, the first product portion and the second product portion defining distinct, non-mixed regions of the oral product.

In at least one example embodiment, the printing the second product portion includes printing the second product portion directly on the first product portion.

In at least one example embodiment, the printing the first product portion includes discharging the first composition from the three-dimensional printer as a liquid and cooling the first composition such that the first product portion is a solid. The printing the second product portion includes discharging the second composition from the three-dimensional printer as a liquid and cooling the second composition such that the second product portion is a solid.

In at least one example embodiment, the cooling the first composition includes circulating air in a region surrounding the first composition.

In at least one example embodiment, the printing the first product portion is completed prior to beginning the printing the second product portion.

In at least one example embodiment, the method further includes preparing the first composition prior to printing the first product portion. The method further includes preparing the second composition prior to printing the second product portion.

In at least one example embodiment, the preparing the first composition includes heating the first composition to a first temperature.

In at least one example embodiment, the first temperature ranges from 150° C. to 160° C.

In at least one example embodiment, the preparing the second composition includes heating the second composition to a second temperature ranging from 150° C. to 160° C.

In at least one example embodiment, the method further comprises preheating the three-dimensional printer. The method further comprises, after the preheating, transferring the first composition from a first vessel to the three-dimensional printer.

In at least one example embodiment, the method preheating the three-dimensional printer. The method further includes, after the preheating, transferring the second composition from a second vessel to the three-dimensional printer.

In at least one example embodiment, the printing the first product portion is performed using a first part of the three-dimensional printer. The printing the second product portion is performed using a second part of the three-dimensional printer.

In at least one example embodiment, the printing the first product portion includes heating a first region of the first part to a first temperature. The printing the second product portion further includes heating a second region of the first part to a second temperature. The second region is downstream of the first region.

In at least one example embodiment, the second temperature is different than the first temperature.

In at least one example embodiment, the second temperature is higher than the first temperature.

In at least one example embodiment, the first temperature ranges from 80° C. to 85° C. The second temperature ranges from 90° C. to 105° C.

In at least one example embodiment, the first region is a first interior region at least partially defined by a syringe. The second region is a second interior region at least partially defined by a nozzle coupled to the syringe.

In at least one example embodiment, the printing the first product portion includes printing a first layer having a first thickness of less than 1 mm.

In at least one example embodiment, the printing the second product portion includes printing a second layer having a second thickness of less than 1 mm.

In at least one example embodiment, the printing the first product portion includes printing a plurality of first product portions. The printing the second product portion includes printing a plurality of second product portions.

In at least one example embodiment, the method further includes printing a third product portion of the oral product. The third product portion has a third composition different from the first composition and the second composition. The first product portion, the second product portion, and the third product portion define distinct, non-mixed regions of the oral product.

In at least one example embodiment, the method further includes controlling a humidity to a desired humidity during the printing the first product portion and the printing the second product portion.

In at least one example embodiment, the desired humidity ranges from 40% to 60%.

At least one example embodiment relates to a method for preparing an oral product.

In at least one example embodiment, the method includes disposing a first composition using a first part of a three-dimensional printer to form one or more dissolvable first portions of the oral product. The method further includes disposing a second composition different from the first composition using a second part of the three-dimensional printer to form one or more dissolvable second portions of the oral product. At least one of the first composition and the second composition include nicotine.

At least one example embodiment relates to an oral product.

In at least one example embodiment, the oral product includes a first portion and a second portion. The first portion has a first composition. The first portion defines a first thickness less than 1 mm. The first portion is homogeneous. The second portion has a second composition. The second portion defines a second thickness less than 1 mm. The second portion is homogeneous. The second portion is directly adjacent to the first portion. The first composition is non-mixed with the second composition.

In at least one example embodiment, at least one of the first composition and the second composition includes nicotine.

In at least one example embodiment, the other of the first composition and the second composition includes a nicotine masking agent.

In at least one example embodiment, the first composition includes a first amount of nicotine. The second composition includes a second amount of nicotine different than the first amount of nicotine.

In at least one example embodiment, the first composition has a first density. The second composition has a second density different than the first density.

In at least one example embodiment, the first portion is a first layer. The second portion is a second layer.

In at least one example embodiment, the oral product is a lozenge.

In at least one example embodiment, the first portion and the second portion are preparing using three-dimensional printing.

At least one example embodiment relates to methods for preparing an oral product.

In at least one example embodiment, the method for preparing an oral product includes disposing a first composition using a first part of a three-dimensional printer to form one or more dissolvable first portions of the oral product and disposing a second composition different from the first composition using a second part of the three-dimensional printer to form one or more dissolvable second portions of the oral product, where at least one of the first composition and the second composition including nicotine.

In at least one example embodiment, the first composition has a first density, and the second composition has a second density that is different from the first density.

In at least one example embodiment, the one or more dissolvable first portions have a first density, and the one or more dissolvable second portions have a second density that is different from the first density.

In at least one example embodiment, the one or more first portions define one or more distinct first layers and the one or more second portions define one or more distinct second layers disposed between or around the one or more distinct first layers to form the oral product.

In at least one example embodiment, the one or more first portions and the one or more second portions together define one or more patterns of the oral product.

In at least one example embodiment, the one or more first portions and the one or more second portions together define one or more patterned layers of the oral product.

In at least one example embodiment, the first composition includes the nicotine, and the second composition includes a masking agent.

In at least one example embodiment, the first composition includes a first amount of the nicotine, and the second composition further includes a second amount of the nicotine, where the first amount of the nicotine is different than the second amount of the nicotine.

In at least one example embodiment, at least one of the one or more first portions further includes a flavorant.

In at least one example embodiment, at least one of the one or more second portions further includes a flavorant.

In at least one example embodiment, the method further includes disposing a third composition using a third part of the three-dimensional printer to form one or more dissolvable third portions of the oral product, where the third composition is different from the first and second compositions.

In at least one example embodiment, the one or more first portions define one or more distinct first layers, the one or more second portions define one or more distinct second layers, and the one or more third portions define one or more distinct third layers, where the one or more distinct first layers, the one or more distinct second layers, and the one or more distinct third layers are alternately disposed to form the oral product.

In at least one example embodiment, the one or more first portions, the one or more second portions, and the one or more third portions together define one or more patterns of the oral product.

In at least one example embodiment, the one or more first portions, the one or more second portions, and the one or more third portions together define one or more patterned layers of the oral product.

In at least one example embodiment, the first composition includes the nicotine, the second composition includes a masking agent, and the third composition includes a flavorant.

In at least one example embodiment, the first composition includes a first amount of the nicotine, the second composition includes a second amount of the nicotine that is different than the first amount, and the third composition includes a third amount of the nicotine that is different than the first amount.

In at least one example embodiment, the third amount is different than the second amount.

At least one example embodiment relates to an oral product.

In at least one example embodiment, the oral product includes one or more first portions, where each of the one or more first portions includes a first material and having a first density, and where the one or more first portions are disposed using a three-dimensional printer. The oral product also includes one or more second portions, where each of the one or more second portions includes a second material different from the first material and having a second density different from the first density, and where the one or more second portions are disposed using the three-dimensional printer.

In at least one example embodiment, the one or more first portions define one or more distinct first layers, and the one or more second portions define one or more distinct second layers disposed between or around the one or more distinct first layers to form the oral product.

### BRIEF DESCRIPTION OF THE DRAWINGS

The various features and advantages of the non-limiting embodiments herein may become more apparent upon review of the detailed description in conjunction with the accompanying drawings. The accompanying drawings are merely provided for illustrative purposes and should not be interpreted to limit the scope of the claims. The accompanying drawings are not to be considered as drawn to scale unless explicitly noted. For purposes of clarity, various dimensions of the drawings may have been exaggerated.

FIG. 1 is a perspective view of an oral product according to at least one example embodiment.

FIG. 2 is a perspective view of another oral product according to at least one example embodiment.

FIG. 3 is a perspective view of another oral product according to at least one example embodiment.

FIG. 4 is a perspective view of an example lozenge according to at least one example embodiment.

FIG. 5 is a top-down view of an example dissolvable film according to at least one example embodiment.

FIG. 6 is a perspective view of an example chewing gum according to at least one example embodiment.

FIG. 7 is a perspective view of an example dissolvable tablet according to at least one example embodiment.

FIG. 8 is a perspective view of an example non-dissolvable chew according to at least one example embodiment.

FIG. 9 is a flow diagram illustrating a method of making an oral product according to at least one example embodiment.

FIG. 10 is a schematic illustration of equipment preparing a composition according to at least one example embodiment.

FIG. 11 is a schematic illustration of a three-dimensional printer according to at least one example embodiment.

FIG. 12 is a flow diagram illustrating another method for preparing an oral product according to at least one example embodiment.

FIG. 13 is a flow diagram illustrating another method for preparing an oral product according to at least one example embodiment.

FIG. 14 is a block diagram illustrating an apparatus for use in preparing an oral product according to at least one example embodiment.

FIG. 15 is a block diagram illustrating another example apparatus for use in preparing an oral product according to at least one example embodiment.

### DETAILED DESCRIPTION OF EXAMPLE EMBODIMENTS

Some detailed example embodiments are disclosed herein. However, specific structural and functional details disclosed herein are merely representative for the purposes of describing example embodiments. Example embodiments may, however, be embodied in many alternate forms and should not be construed as limited to only the example embodiments set forth herein.

Accordingly, while example embodiments are capable of various modifications and alternative forms, example embodiments thereof are shown by way of example in the drawings and will herein be described in detail. It should be understood, however, that there is no intent to limit example embodiments to the particular forms disclosed, but to the contrary, example embodiments are to cover all modifications, equivalents, and alternatives falling within the scope of example embodiments. Like numbers refer to like elements throughout the description of the figures.

It should be understood that when an element or layer is referred to as being “on,” “connected to,” “coupled to,” or “covering” another element or layer, it may be directly on, connected to, coupled to, or covering the other element or layer or intervening elements or layers may be present. In contrast, when an element is referred to as being “directly on,” “directly connected to,” or “directly coupled to” another element or layer, there are no intervening elements or layers present. Like numbers refer to like elements throughout the specification. As used herein, the term “and/or” includes any and all combinations of one or more of the associated listed items.

It should be understood that, although the terms first, second, third, etc. may be used herein to describe various elements, regions, layers and/or sections, these elements, regions, layers, and/or sections should not be limited by these terms. These terms are only used to distinguish one element, region, layer, or section from another region, layer, or section. Thus, a first element, component, region, layer, or section discussed below could be termed a second element, region, layer, or section without departing from the teachings of example embodiments.

Spatially relative terms (e.g., “beneath,” “below,” “lower,” “above,” “upper,” and the like) may be used herein for ease of description to describe one element or feature's relationship to another element(s) or feature(s) as illustrated in the figures. It should be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, the term “below” may encompass both an orientation of above and below. The device may be otherwise oriented (rotated 90 degrees or at other orientations) and the spatially relative descriptors used herein interpreted accordingly.

The terminology used herein is for the purpose of describing various example embodiments only and is not intended to be limiting of example embodiments. As used herein, the singular forms “a,” “an,” and “the” are intended to include the plural forms as well, unless the context clearly indicates otherwise. It will be further understood that the terms “includes,” “including,” “comprises,” and/or “comprising,” specify the presence of stated features, integers, steps, operations, and/or elements, but do not preclude the presence or addition of one or more other features, integers, steps, operations, elements, and/or groups thereof.

When the terms “about” or “substantially” are used in this specification in connection with a numerical value, it is intended that the associated numerical value includes a manufacturing or operational tolerance (e.g., ±10%) around the stated numerical value. Moreover, when the terms “generally” or “substantially” are used in connection with geometric shapes, it is intended that precision of the geometric shape is not required but that latitude for the shape is within the scope of the disclosure. Furthermore, regardless of whether numerical values or shapes are modified as “about,” “generally,” or “substantially,” it will be understood that these values and shapes should be construed as including a manufacturing or operational tolerance (e.g., ±10%) around the stated numerical values or shapes.

Unless otherwise defined, all terms (including technical and scientific terms) used herein have the same meaning as commonly understood by one of ordinary skill in the art to which example embodiments belong. It will be further understood that terms, including those defined in commonly used dictionaries, should be interpreted as having a meaning that is consistent with their meaning in the context of the relevant art and will not be interpreted in an idealized or overly formal sense unless expressly so defined herein.

The present disclosure provides oral products and three-dimensional printing methods of forming the same.

In at least one example embodiment, the oral product is an oral tobacco product, an oral non-tobacco product, an oral cannabis product, or any combination thereof.

In at least one example embodiment, the oral product includes a tobacco extract, such as a tobacco-derived nicotine extract and/or synthetic nicotine. The oral product may include the tobacco-derived nicotine extract and/or synthetic nicotine alone or in combination with a carrier. The carrier may be a non-tobacco material (e.g., microcrystalline cellulose) or a tobacco material (e.g., tobacco fibers having reduced or eliminated nicotine content, which may be referred to as “exhausted tobacco plant tissue or fibers”). In at least one example embodiment, the exhausted tobacco plant tissue or fibers can be treated to remove at least 25% (e.g., at least 40%, at least 50%, at least 60%, at least 70%, at least 75%, at least 80%, at least 85%, at least 90%, or at least 95%) of the nicotine. The tobacco plant tissue may be, for example, washed with water or another solvent to remove the nicotine

In at least one example embodiment, the oral product includes, additionally, or alternatively, nicotine or a nicotine derivative. For example, the oral product may include a complex of nicotine or a salt of nicotine. The complex of nicotine may include, for example, nicotine polacrilex. The salt of nicotine may include, for example, nitrate, monotartrate, bitartrate, bitartrate dihydrate, salicylate, sulfate or bisulfate, phosphate or acid phosphate, acetate, lactate, succinate, maleate, fumarate, gluconate, saccharate, benzoate, methanesulfonate, hydrochloride, hydrobromide, hydroiodide, or any combination thereof.

In at least one example embodiment, the oral product includes the nicotine or nicotine derivative in an amount greater than or equal to about 0.1 milligram (e.g., greater than or equal to about 1 milligram, greater than or equal to about 2 milligrams, greater than or equal to about 4 milligrams, or greater than or equal to about 6 milligrams, greater than or equal to about 8 milligrams, greater than or equal to about 10 milligrams, greater than or equal to about 12 milligrams). The oral product may include the nicotine or nicotine derivative in an amount less than or equal to about 14 milligrams (e.g., less than or equal to about 12 milligrams, less than or equal to about 10 milligrams, less than or equal to about 8 milligrams, less than or equal to about 6 milligrams, less than or equal to about 4 milligrams, less than or equal to about 2 milligrams, or less than or equal to about 1 milligram). In at least one example embodiment, the oral product includes the nicotine or nicotine derivative in an amount ranging from about 3 milligrams to about 8 milligrams.

In at least one example embodiment, the oral product includes, additionally, or alternatively, cannabis, such as cannabis plant tissue and/or cannabis extracts. The cannabis material may include, for example, leaf and/or flower material from one or more species of cannabis plants and/or extracts from the one or more species of cannabis plants. The one or more species of cannabis plants may include Cannabis sativa, Cannabis indica, and/or Cannabis ruderalis. In at least one example embodiment, the cannabis may be in the form of fibers. In at least one example embodiment, the cannabis may include a cannabinoid, a terpene, and/or a flavonoid. In at least one example embodiment, the cannabis material may be a cannabis-derived cannabis material, such as a cannabis-derived cannabinoid, a cannabis-derived terpene, and/or a cannabis-derived flavonoid.

In at least one example embodiment, the oral product includes, additionally, or alternatively, one or more compounds or elements or components such as a mouth-stable polymer, a mouth-soluble polymer, a sweetener, an energizing agent, a soothing agent, a focusing agent, a plasticizer, mouth-soluble fibers, an alkaloid, a mineral, a vitamin, a dietary supplement, a nutraceutical, a coloring agent, an amino acid, a sensate or chemesthetic agent, an antioxidant, a food-grade emulsifier, a pH modifier, a botanical, a tooth-whitening agent, a therapeutic agent, a processing aid, a stearate, a wax, a stabilizer, a disintegrating agent, a lubricant, a preservative, a filler, a flavorant, flavor masking agents, a bitterness receptor site blocker, a receptor site enhancers, a carrier, an oil, a base polymer, a masking agent, one or more additives, an effervescent, or any combination thereof.

As used herein, “mouth-soluble” means that the polymer experiences significant degradation when exposed to saliva within an oral cavity of an adult consumer over a period of about four hours. In at least one example embodiment, the mouth-soluble polymer disintegrates when exposed to saliva having a normal human body temperature (i.e., 98.6° F.) for a period of less than or equal to about an hour (e.g., less than or equal to about 30 minutes, less than or equal to about 15 minutes, less than or equal to about 10 minutes, or less than or equal to about 5 minutes).

In at least one example embodiment, the oral product is free of a mouth soluble polymer. In at least one example embodiment, the oral product includes a mouth soluble polymer. The mouth-soluble polymer may include, for example, a cellulosic polymer, a natural polymer, a seaweed-derived polymer, a microbial-derived polymer, an extract, an exudate, a synthetic polymer, or any combination thereof. Other useful mouth-soluble polymers are known in the art, for example, see Krochta et al., Food Technology (1997) at 51:61-74; Glicksman Food Hydrocolloids CRC 1982; Krochta Edible Coatings and Films to Improve Food Quality Technomic 1994; Industrial Gums Academic 1993; and/or Nussinovitch Water-Soluble Polymer Applications in Foods Blackwell Science 2003, the entire contents of which are hereby incorporated by reference.

In at least one example embodiment, the cellulosic polymer may include, for example, carboxymethyl cellulose (CMC), hydroxypropyl (HPC), hydroxyethyl cellulose (HEC), hydroxypropyl methylcellulose (HPMC), methyl cellulose (MC), or any combination thereof. In at least one example embodiment, the natural polymer may include, for example, a starch, a modified starch, konjac, collagen, inulin, soy protein, whey protein, casein, wheat gluten, or any combination thereof. In at least one example embodiment, the seaweed-derived polymer may include, for example, a carrageenan, an alginate, or a combination of a carrageenan and an alginate. In at least one example embodiment, the carrageenan may include, for example, kappa carrageenan, iota carrageenan, lambda carrageenan, or any combination thereof. In at least one example embodiment, the alginate may include, for example, propylene glycol alginate. In at least one example embodiment, the microbial-derived polymer may include, for example, xanthan, dextran, pullulan, curdlan, gellan, or any combination thereof. In at least one example embodiment, the extract may include, for example, locust bean gum, guar gum, tara gum, gum tragacanth, pectin (e.g., low methoxy and amidated), agar, zein, karaya, gelatin, psyllium seed, chitin, chitosan, or any combination thereof. In at least one example embodiment, the exudate may include, for example, gum acacia (arabic), shellac, or any combination thereof. In at least one example embodiment, the synthetic polymer may include, for example, polyvinyl pyrrolidone, polyethylene oxide, polyvinyl alcohol, or any combination thereof.

As used herein, “mouth stable” means that the polymer does not appreciably dissolve or disintegrate when exposed to saliva at the normal human body temperature (i.e., 98.6° F.) over a period of about one hour. In at least one example embodiment, the mouth-stable polymer is a biodegradable polymer that is configured to break down over a period of days, weeks, months, or years but does not appreciably break down when held in an oral cavity and exposed to saliva for a period of about one hour. In at least one example embodiment, the mouth-stable polymer is stable within an oral cavity and exposed to saliva at the normal human body temperature for a period of greater than or equal to about 2 hours (e.g., greater than or equal to about 6 hours, greater than or equal to about 12 hours, greater than or equal to about 1 day, or greater than or equal to about 2 days). Accordingly, an oral product including a mouth-stable polymer according to at least one example embodiment is configured to remain intact when placed in an adult consumer's mouth. After a period of time, the mouth-stable polymer and any other mouth-stable elements may be removed from the adult consumer's mouth and discarded.

The mouth-stable polymer may be biocompatible and biostable. The mouth-stable polymer may generally be recognized as safe and in compliance with applicable food-contact regulations by an appropriate regulatory agency (e.g., the U.S. Food and Drug Administration (FDA)). In at least one example embodiment, the mouth-stable polymer has a flexural modulus of greater than or equal to about 5 MPa (e.g., greater than or equal to about 10 MPa) when tested according to ASTM Testing Method D790 or ISO 178 at 23° C.

In at least one example embodiment, the mouth-stable polymer may include, for example, a polyurethane, a silicone, a polyester, a polyacrylate, a polyethylene, a polypropylene, a polyetheramide, a polystyrene, a polyvinyl alcohol, a polyvinyl acetate, a polyvinyl chloride, a polybutyl acetate, a butyl rubber, poly(styrene-ethylene-butylene-styrene) (SEBS), poly(styrene-butadiene-styrene) (SBS), poly(styrene-isoprene-styrene) (SIS), any copolymer thereof, or any combination thereof. In at least one example embodiment, the mouth-stable polymer includes a food-grade or medical-grade polymer, such as medical-grade polyurethane.

In at least one example embodiment, the mouth-stable polymer includes, for example, a thermoplastic polymer. The thermoplastic polymer may include a thermoplastic elastomer. In at least one example embodiment, the mouth-stable polymer includes a thermoplastic elastomer meeting the requirements of the FDA-modified ISO 10993, Part 1 “Biological Evaluation of Medical Devices” tests with human tissue contact time of 30 days or less. In at least one example embodiment, the mouth-stable polymer has a shore Hardness of 50D or softer, a melt flow index of about 3 g/10 min at 200° C./10 kg, a tensile strength of greater than or equal to about 10 MPa (using ISO 37), and/or an ultimate elongation of less than about 100% (using ISO 37).

In at least one example embodiment, the oral product is free of a sweetener. In at least one example embodiment, the oral product includes a sweetener. In at least one example embodiment, the oral product includes an encapsulated sweetener. The sweetener may include a synthetic sweetener, a natural sweetener, or a combination of a synthetic sweetener and a natural sweetener.

The natural sweetener may include, for example, a sugar such as monosaccharide, a disaccharide, a polysaccharide, or any combination thereof. The natural sweetener may include, for example, sucrose, honey, a mixture of low-molecular-weight sugars excluding sucrose, glucose (i.e., grape sugar, corn sugar, dextrose), molasses, corn sweetener, glucose syrup (i.e., corn syrup), fructose (i.e., fruit sugar), lactose (i.e., milk sugar), maltose (i.e., malt sugar, maltobiose), sorghum syrup, fruit juice concentrate, or any combination thereof.

In at least one example embodiment, the sweetener includes a sugar alcohol. The sugar alcohol may include, for example, ethylene glycol, glycerol, erythritol, threitol, arabitol, xylitol, ribitol, mannitol, sorbitol, galactitol, fucitol, iditol, inositol, volemitol, isomalt, maltitol, lactitol, maltotriitol, maltotetraitol, polyglycitol, or any combination thereof. In at least one example embodiment, the sweetener includes a non-nutritive sweetener. The non-nutritive sweetener may include, for example, stevia, saccharin, aspartame, sucralose, acesulfame potassium, or any combination thereof.

In at least one example embodiment, the oral product is free of an energizing agent. In at least one example embodiment, the oral product includes an energizing agent. The energizing agent may include, for example, caffeine, taurine, citicoline, glucaronalactone, vitamin B6, vitamin B12, guarana, or any combination thereof. The caffeine may include, for example, synthetic caffeine, natural caffeine, or a combination of synthetic caffeine and natural caffeine.

In at least one example embodiment, the oral product may include the energizing agent (for example, the caffeine) in amount greater than or equal to about 10 milligrams (e.g., greater than or equal to about 25 milligrams, greater than or equal to about 50 milligrams, greater than or equal to about 75 milligrams, greater than or equal to about 100 milligrams, greater than or equal to about 150 milligrams). In at least one example embodiment, the oral product may include the caffeine in an amount less than or equal to about 200 milligrams (e.g., less than or equal to about 150 milligrams, less than or equal to about 100 milligrams, less than or equal to about 75 milligrams, less than or equal to about 50 milligrams, or less than or equal to about 25 milligrams).

In at least one example embodiment, the oral product may include the energizing agent (for example, the caffeine) in amount greater than or equal to about 0.1 weight percent (e.g., greater than or equal to about 0.5 weight percent, greater than or equal to about 1 weight percent, greater than or equal to about 1.5 weight percent, greater than or equal to about 2 weight percent, greater than or equal to about 2.5 weight percent, greater than or equal to about 3 weight percent, greater than or equal to about 3.5 weight percent, greater than or equal to about 4 weight percent, or greater than or equal to about 4.5 weight percent). In at least one example embodiment, the oral product may include the energizing agent (for example, the caffeine) in amount less than or equal to about 5 weight percent (e.g., less than or equal to about 4.5 weight percent, less than or equal to about 4 weight percent, less than or equal to about 3.5 weight percent, less than or equal to about 3 weight percent, less than or equal to about 2.5 weight percent, less than or equal to about 2 weight percent, less than or equal to about 1.5 weight percent, less than or equal to about 1 weight percent, or less than or equal to about 0.5 weight percent).

As used herein, “soothing agents” are those selected to provide a soothing sensation to the throat and/or oral cavity of an adult consumer. In at least one example embodiment, the oral product is free of a soothing agent. In at least one example embodiment, the oral product includes a soothing agent. The soothing agent may include, for example, chamomile, lavender, jasmine, theanine, melatonin, or both theanine and melatonin.

In at least one example embodiment, the oral product includes the soothing agent in an amount greater than or equal to about 0.1 weight percent (e.g., greater than or equal to about 0.5 weight percent, greater than or equal to about 1 weight percent, greater than or equal to about 1.5 weight percent, greater than or equal to about 2 weight percent, greater than or equal to about 2.5 weight percent, greater than or equal to about 3 weight percent, greater than or equal to about 3.5 weight percent, greater than or equal to about 4 weight percent, or greater than or equal to about 4.5 weight percent). In at least one example embodiment, the oral product includes the soothing agent in an amount less than or equal to about 5 weight percent (e.g., less than or equal to about 4.5 weight percent, less than or equal to about 4 weight percent, less than or equal to about 3.5 weight percent, less than or equal to about 3 weight percent, less than or equal to about 2.5 weight percent, less than or equal to about 2 weight percent, less than or equal to about 1.5 weight percent, less than or equal to about 1 weight percent, or less than or equal to about 0.5 weight percent).

In at least one example embodiment, the oral product is free of a focusing agent. In at least one example embodiment, the oral product includes a focusing agent. The focusing agent may include, for example, Ginkgo biloba.

In at least one example embodiment, the oral product is free of a plasticizer. In at least one example embodiment, the oral product includes a plasticizer. The plasticizer may include, for example, a triglyceride (e.g., long, medium, and/or short chain), triacetin, propylene glycol, glycerin, vegetable oil, a phthalate, an ester of a polycarboxylic acid with a linear or branched aliphatic alcohol of moderate chain length, or any combination thereof. In at least one example embodiment, the plasticizer may be present in addition to triglycerides and/or other oils in the liquid mixture.

In at least one example embodiment, the oral product is free of a mouth-soluble fiber. In at least one example embodiment, the oral product includes a mouth-soluble fiber. In at least one example embodiment, the mouth-soluble fibers include maltodextrin, psyllium, starch, or any combination thereof. In at least one example embodiment, the mouth-soluble fibers include soluble dietary fibers. In at least one example embodiment oral product includes partially-soluble fibers, such as sugar beet fibers.

In at least one example embodiment, the oral product is free of minerals in addition to any that may be present due to the inclusion, for example, of fruits and/or vegetables. In at least one example embodiment, the oral product includes minerals in addition to any that may be present due to the inclusion, for example, of fruits and/or vegetables. The minerals may include, for example, calcium, magnesium, phosphorus, iron, zinc, iodine, selenium, potassium, copper, manganese, molybdenum, chromium, and any combination thereof. The amount of minerals incorporated into the oral product for adult human consumption can be varied according to the type of mineral and the intended adult consumer. The amount of minerals may be formulated to include an amount less than or equal to the recommendations of the United States Department of Agriculture Recommended Daily Allowances.

In at least one example embodiment, the oral product may be free of vitamins in addition to any that may be present due to the inclusion, for example, of fruits and/or vegetables. In at least one example embodiment, the oral product may include one or more vitamins in addition to any that may be present due to the inclusion, for example, of fruits and/or vegetables. The one or more additional vitamins may include, for example, vitamin A (retinol), vitamin D (cholecalciferol), vitamin E group, vitamin K group (phylloquinones and menaquinones), thiamine (vitamin B1), riboflavin (vitamin B2), niacin, niacinamide, pyridoxine (vitamin B6 group), folic acid, choline, inositol, vitamin B12 (cobalamins), PABA (para aminobezoic acid), biotin, vitamin C (ascorbic acid), and any combination thereof. The amount of vitamins may be chosen so as to provide an amount less than or equal to the recommendations of the United States Department of Agriculture Recommended Daily Allowances.

As used herein, the term “nutraceuticals” refers to any ingredient in foods that has a beneficial effect on human health. In at least one example embodiment, the oral product is free of nutraceuticals. In at least one example embodiment, the oral product includes a nutraceutical. The nutraceutical may include, for example, particular compounds and/or compositions isolated from natural food sources and/or genetically modified food sources. The nutraceutical may include, for example, phytonutrients derived from natural plants and/or genetically engineered plants.

In at least one example embodiment, the oral product includes the nutraceutical in an amount greater than or equal to about 0.1 weight percent (e.g., greater than or equal to about 0.5 weight percent, greater than or equal to about 1 weight percent, greater than or equal to about 1.5 weight percent, greater than or equal to about 2 weight percent, greater than or equal to about 2.5 weight percent, greater than or equal to about 3 weight percent, greater than or equal to about 3.5 weight percent, greater than or equal to about 4 weight percent, or greater than or equal to about 4.5 weight percent). In at least one example embodiment, the oral product includes the nutraceutical in an amount less than or equal to about 5 weight percent (e.g., less than or equal to about 4.5 weight percent, less than or equal to about 4 weight percent, less than or equal to about 3.5 weight percent, less than or equal to about 3 weight percent, less than or equal to about 2.5 weight percent, less than or equal to about 2 weight percent, less than or equal to about 1.5 weight percent, less than or equal to about 1 weight percent, or less than or equal to about 0.5 weight percent).

In at least one example embodiment, the oral product is free of a coloring agent. In at least one example embodiment, the oral product includes a coloring agent. The coloring agent may include a natural colorant, an artificial colorant, or any combination thereof.

In at least one example embodiment, the oral product is free of a sensate or chemesthesis agent. In at least one example embodiment, the oral product includes a sensate or chemesthesis agent. The sensate or chemesthesis agent may includes any soothing, cooling, and/or warming agent. In at least one example embodiment, the sensate or chemesthesis agent includes, for example, mint, menthol, cinnamon, pepper, jambu, capsaicin, pipeline, alpha-hydroxy-sanshool, (8)-gingerole, menthol, menthyl lactate, WS-3 (N-Ethyl-p-menthane-3-carboxamide), WS-23 (2-Isopropyl N,2,3-trimethylbutyramide), Evercool 180™ (available from Givaudan SA), or any combination thereof. In at least one example embodiment, the sensate or chemesthesis agent includes, for example, capsaicin, pipeline, alpha-hydroxy-sanshool, (8)-gingerole, or any combination thereof, which are selected to provide a warm, tingling or burning sensation. In at least one example embodiment, the sensate or chemesthesis agent includes, for example, menthol, menthyl lactate, WS-3 (N-Ethyl-p-menthane-3-carboxamide), WS-23 (2-Isopropyl N,2,3-trimethylbutyramide), Evercool 180™ (available from Givaudan SA), or any combination thereof, which are selected to provide a cooling sensation.

In at least one example embodiment, the oral product is free of an amino acid. In at least one example embodiment, the oral product includes an amino acid. The amino acid may include, for example, the eight essential amino acids that cannot be biosynthetically produced in humans, including valine, leucine, isoleucine, lysine, threonine, tryptophan, methionine, phenylalanine, or any combination thereof. The amino acid may include, for example, non-essential amino acids, such as alanine, arginine, asparagine, aspartic acid, cysteine, glutamic acid, glutamine, glycine, histidine, proline, serine, tyrosine, and any combination thereof.

In at least one example embodiment, the oral product includes the amino acid in an amount greater than or equal to about 0.1 weight percent (e.g., greater than or equal to about 0.5 weight percent, greater than or equal to about 1 weight percent, greater than or equal to about 1.5 weight percent, greater than or equal to about 2 weight percent, greater than or equal to about 2.5 weight percent, greater than or equal to about 3 weight percent, greater than or equal to about 3.5 weight percent, greater than or equal to about 4 weight percent, or greater than or equal to about 4.5 weight percent). In at least one example embodiment, the oral product includes the amino acid in an amount less than or equal to about 5 weight percent (e.g., less than or equal to about 4.5 weight percent, less than or equal to about 4 weight percent, less than or equal to about 3.5 weight percent, less than or equal to about 3 weight percent, less than or equal to about 2.5 weight percent, less than or equal to about 2 weight percent, less than or equal to about 1.5 weight percent, less than or equal to about 1 weight percent, or less than or equal to about 0.5 weight percent).

In at least one example embodiment, the oral product is free of a pH modifier. In at least one example embodiment, the oral product includes a pH modifier. The pH modifier may include, for example, ammonium carbonate, ammonium bicarbonate, ammonium hydroxide, calcium carbonate, potassium carbonate, potassium bicarbonate, potassium hydroxide, sodium carbonate, sodium bicarbonate, sodium hydroxide, or any combination thereof.

In at least one example embodiment, the oral product includes the pH adjuster in an amount less than or equal to about 2 weight percent (e.g., less than or equal to about 1 weight percent, less than or equal to about 0.5 weight percent, less than or equal to about 0.1 weight percent, or less than or equal to about 0.05 weight percent). In at least one example embodiment, the oral product includes the pH adjuster in an amount ranging from about 0.01 weight percent to 2 weight percent.

In at least one example embodiment, the oral product is free of a wax. In at least one example embodiment, the oral product includes a wax. The wax may include, for example, paraffin, microcrystalline wax, or both paraffin and microcrystalline wax.

In at least one example embodiment, the oral product is free of a filler. In at least one example embodiment, the oral product includes a filler. The filler may be configured to alter a texture or pliability of the oral product. The filler may include mouth-soluble elements, mouth-insoluble elements, or both mouth-soluble and mouth-insoluble elements. Mouth-soluble elements may be configured to dissolve or disintegrate when in an adult consumer's mouth so as to render the oral product more pliable. The filler may include, for example, dicalcium phosphate, calcium sulfate, a clay, silica, glass particles, glyceryl palmitostearate, sodium stearyl fumarate, talc, or any combination thereof. In at least one example embodiment, certain other compounds or elements or components, including, for example, mouth-soluble fibers, sweeteners, minerals, as described, may be classified as fillers. For example, in at least one example embodiment, cellulosic materials may be present in the oral product as fillers in addition to or as an alternative to being carriers for the liquid mixture.

In at least one example embodiment, the oral product includes the filler in an amount less than or equal to 20 weight percent (e.g., less than or equal to 15 weight percent, less than or equal to 10 weight percent, less than or equal to 9 weight percent, less than or equal to 8 weight percent, less than or equal to 7 weight percent, less than or equal to 6 weight percent, less than or equal to 5 weight percent, less than or equal to 4 weight percent, less than or equal to 3 weight percent, less than or equal to 2 weight percent, or less than or equal to 1 weight percent). In at least one example embodiment, the oral product includes the filler in an amount greater than or equal to about 0 weight percent (e.g., greater than or equal to about 1 weight percent, greater than or equal to about 2 weight percent, greater than or equal to about 3 weight percent, greater than or equal to about 4 weight percent, or greater than or equal to about 5 weight percent). In at least one example embodiment, the oral product includes the filler in an amount ranging from about 0 weight percent to about 8 weight percent.

In at least one example embodiment, the oral product is free of a flavorant. In at least one example embodiment, the oral product includes a flavorant. In at least one example embodiment, the oral product includes an encapsulated flavorant. The flavorant may be natural or artificial. The flavorant may include, for example, a fruit flavorant (e.g., bergamot, berry, cherry, lemon, and/or orange), a liquor or liqueur flavorant (e.g., bourbon, cognac, scotch, whiskey, and/or DRAMBUIE brand liqueur), a mint flavorant (e.g., Japanese mint, menthol, peppermint, spearmint, wintergreen, and/or mint oils from a species of the genus Mentha), a floral flavorant (e.g., geranium, lavender, and/or rose), a spice, an herb, or another botanical or botanical-derived flavorant (e.g., anise, Apium graveolens, caraway, cardamom, cascarilla, cassia, cinnamon, chamomile, clove, cocoa, coffee, coriander, fennel, ginger, jasmine, licorice, nutmeg, pimenta, sage, sandalwood vanilla, and/or ylang-ylang), honey essence, or any combination thereof. In at least one example embodiment, the flavorant includes bergamot, berry, cherry, lemon, orange, bourbon, cognac, scotch, whiskey, DRAMBUIE brand liqueur, Japanese mint, menthol, peppermint, spearmint, wintergreen, mint oils from a species of the genus Mentha, geranium, lavender, rose, anise, Apium graveolens, caraway, cardamom, cascarilla, cassia, cinnamon, chamomile, clove, coffee, coriander, fennel, ginger, jasmine, licorice, nutmeg, pimenta, sage, sandalwood vanilla, ylang-ylang, honey essence, or any combination thereof.

In at least one example embodiment, the oral product is free of a carrier. In at least one example embodiment, the oral product includes a carrier. The carrier may include a liquid carrier. The liquid carrier may include, for example, water, propylene, glycol, glycerin, ethanol, or any combination thereof.

In at least one example embodiment, the oral product is free of an oil. In at least one example embodiment, the oral product includes an oil. The oil may include, for example, a triglyceride, triacetin, a flavor oil, or any combination thereof. The flavor oil may be or may include a flavorant.

An amount of oil may be selected to achieved a desired texture and/or softness. For example, the softness of the oral product may be increased as an amount of the oil in the oral product increases. In at least one example embodiment, the oral product includes oil in an amount greater than or equal to about 5 weight percent (e.g., greater than or equal to about 8 weight percent, greater than or equal to about 9 weight percent, greater than or equal to about 10 weight percent, greater than or equal to about 11 weight percent, greater than or equal to about 12 weight percent, greater than or equal to about 13 weight percent, or greater than or equal to about 15 weight percent). In at least one example embodiment, the oral product includes oil in an amount less than or equal to about 20 weight percent (e.g., less than or equal to about 15 weight percent, less than or equal to about 14 weight percent, less than or equal to about 13 weight percent, less than or equal to about 12 weight percent, less than or equal to about 11 weight percent, less than or equal to about 10 weight percent, or less than or equal to about 8 weight percent). In at least one example embodiment, the oral product includes the oil in an amount ranging from about 8 weight percent to about 16 weight percent (e.g., about 10 weight percent to about 14 weight percent, about 11 weight percent to about 13 weight percent, or about 12 weight percent).

In at least one example embodiment, the oral product is free of a base polymer. In at least one example embodiment, the oral product includes a base polymer. The gum base polymer may include a single polymer or a combination of polymers. In at least one example embodiment, the base polymer includes a resin, an elastomer, or a combination of a resin and an elastomer. The resin may include, for example, polyvinyl acetate (PVA), glycerol esters of gum, terpene resins, or any combination thereof. The elastomer may include, for example, Couma macrocarpa, loquat, tunu, jelutong, chicle, styrene-butadiene rubber, butyl rubber, polyisobutylene, or any combination thereof. In at least one example embodiment, the base polymer includes a natural latex, a vegetable gum (e.g., chicle), a spruce gum, a mastic gum, or any combination thereof.

In at least one example embodiment, the oral product may include the base polymer an amount greater than or equal to about 30 weight percent (e.g., greater than or equal to about 35 weight percent, greater than or equal to about 40 weight percent, greater than or equal to about 45 weight percent, greater than or equal to about 50 weight percent, or greater than or equal to about 55 weight percent). In at least one example embodiment, the oral product may include the base polymer in an amount less than or equal to about 60 weight percent (e.g., less than or equal to about 55 weight percent, less than or equal to about 50 weight percent, less than or equal to about 45 weight percent, or less than or equal to about 40 weight percent). In at least one example embodiment, the oral product includes the base polymer in an amount ranging from about 35 weight percent to about 55 weight percent (e.g., about 40 weight percent to about 50 weight percent, about 43 weight percent to about 47 weight percent, about 44 weight percent to about 46 weight percent, or about 45 weight percent).

In at least one example embodiment, the oral product is free of an effervescent. In at least one example embodiment, the oral product includes an effervescent. The effervescent includes, for example, carbon dioxide, which may be embedded, for example, in a flavorant.

The oral product may have various ranges of moisture. In at least one example embodiment, the oral product is a dry oral product having a moisture content ranging from about 5 percent by weight to about 10 percent by weight. In at least one example embodiment, the oral product has a medium moisture content, such as a moisture content ranging from about 20 percent by weight to about 35 percent by weight. In at least one example embodiment, the oral product is a wet oral product having a moisture content ranging from about 40 percent by weight to about 55 percent by weight.

The oral product may be in the form, for example, of shaped material (e.g., plugs or twists), tablets, lozenges, chews, gums, films, or any combination thereof. The oral product may be manufactured to have a variety of different sizes and/or shapes and/or configurations. The oral product may be sized and/or shaped and/or configured to be wholly received in an oral cavity of an adult tobacco consumer. The oral product may have an oval shape, a rounded shield shape, a flat shield shape an elliptical shape, an elongated elliptical shape, a semi-circular shape, a square shape, a rounded-edge square shape, a rectangular shape, an elongated rectangular shape, a rounded-edge rectangular shape, a football shape, a boomerang shape, a teardrop shape, a comma shape, a bowtie shape, a peanut shape, or any combination thereof. The oral product may have an oral-shaped cross section, a rectangular cross section, an elongated rectangular cross section, a lens or football shaped cross section, a boomerang-shaped cross section, a shield-shaped cross section, or any combination thereof. In at least one example embodiment, the size and/or shape and/or configuration of the oral product may be selected to promote desired positioning of the oral product within the oral cavity and/or packaging.

In at least one example embodiment, the oral product may have dimensions ranging from about 1 millimeter (mm) to about 25 mm (e.g., about 1 mm to about 10 mm, about 1 mm to about 5 mm, about 5 mm to about 25 mm, about 5 mm to about 10 mm, about 10 mm to about 15 mm, about 15 mm to about 20 mm, or about 20 mm to about 25 mm). In at least one example embodiment, the oral product has a first dimension (e.g., smallest dimension or thickness) ranging from about 1 mm to about 10 mm (e.g., about 2.5 mm). In at least one example embodiment, the oral product has a largest dimension (e.g., diameter, height, or width) ranging from about 5 mm to about 25 mm (e.g., about 12 mm).

In at least one example embodiment, the oral product may have a weight ranging from about 1 gram to about 10 grams (e.g., about 1 gram to about 9 grams, about 1 gram to about 8 grams, about 1 gram to about 7 grams, about 1 gram to about 6 grams, about 1 gram to about 5 grams, about 1 gram to about 4 grams, about 1 gram to about 3 grams, about 1 gram to about 2 grams, about 2 grams to about 9 grams, about 2 grams to about 8 grams, about 2 grams to about 7 grams, about 2 grams to about 6 grams, about 2 grams to about 5 grams, about 2 grams to about 4 grams, about 2 grams to about 3 grams, about 3 grams to about 9 grams, about 3 grams to about 8 grams, about 3 grams to about 7 grams, about 3 grams to about 6 grams, about 3 grams to about 5 grams, about 3 grams to about 4 grams, about 4 grams to about 9 grams, about 4 grams to about 8 grams, about 4 grams to about 7 grams, about 4 grams to about 6 grams, about 4 grams to about 5 grams, about 5 grams to about 9 grams, about 5 grams to about 8 grams, about 5 grams to about 7 grams, about 5 grams to about 6 grams, about 6 grams to about 9 grams, about 6 grams to about 8 grams, about 6 grams to about 7 grams, about 7 grams to about 9 grams, about 7 grams to about 8 grams, about 8 grams to about 9 grams).

In at least one example embodiment, the oral product includes a uniform unitary structure. In at least one example embodiment, the oral product includes two or more continuous or distinct layers. The two or more layers may be separable or inseparable. Each of the two or more layers may have the same or different compositions. For example, in at least one example embodiment, the two or more layers may have different flavors, different amounts of nicotine or nicotine derivative (including no nicotine or nicotine derivative), textures, softnesses, different release relates, or any combination thereof.

In at least one example embodiment, the oral product may include two or more distinct portions or regions and/or one or more distinct patterns. For example, in at least one example embodiment, the oral product may include one or more first layers and one or more second layers disposed around or between the one or more first layers.

In at least one example embodiment, the oral product may include one or more coatings. The one or more coatings may include any of the described materials. Each of the one or more coatings may include the same or different compositions and structure. For example, in at least one example embodiment, one of the one or more coatings may include a mouth-soluble polymer. In at least one example embodiment, one of the one or more coatings may include a nicotine or a nicotine derivative. In at least one example embodiment, one of the one or more coatings may include a flavorant, a sweetener, or any combination thereof. The weight percentages described herein refer to an uncoated oral product or a body of a coated oral product.

Each of the one or more coatings may partially or fully surround a body of the oral product. In at least one example embodiment, each of the one or more coatings may cover greater than or equal to about 20% of a total surface area of the body (e.g., greater than or equal to about 25%, greater than or equal to about 30%, greater than or equal to about 35%, greater than or equal to about 40%, greater than or equal to about 45%, greater than or equal to about 50%, greater than or equal to about 55%, greater than or equal to about 60%, greater than or equal to about 65%, greater than or equal to about 70%, greater than or equal to about 75%, greater than or equal to about 80%, greater than or equal to about 85%, greater than or equal to about 90%, greater than or equal to about 95%, greater than or equal to about 98%, greater than or equal to about 99%, or greater than or equal to about 99.5%).

In at least one example embodiment, the oral product may include a functional and/or decorative indicia. The indicia may include, for example, a trademark, a product name, an image, a flavor indicator, a decoration, a date, a product identifier, a manufacturer identifier, a lot number, or any combination thereof. In at least one example embodiment, the indicia are embossed or debossed on an exterior surface of the oral product. In at least one example embodiment, the indicia are in the form of a dissolvable film on the exterior surface of the oral product.

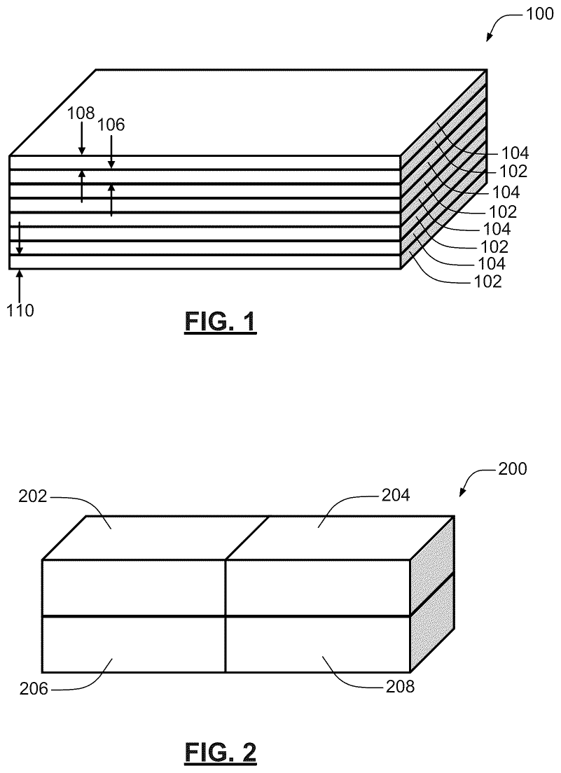

FIG. 1 is a perspective view of an oral product according to at least one example embodiment.

In at least one example embodiment, as shown in FIG. 1, an oral product 100 includes one or more first product portions or regions 102 and one or more second product portions or regions 104. The first product portion 102 has a first composition. The second product portion 104 has a second composition. The first and second compositions are different. In at least one example embodiment, the first product portion 102 has a first density and the second product portion has a second density that is different from the first density.

In at least one example embodiment, the first and second product portions 102, 104 are in distinct regions having the first and second compositions, respectively. The second product portion 104 may be in direct contact with the first product portion 102 such that the oral product 100 is free of a mixed composition layer between the first and second product portions 102, 104.

In at least one example embodiment, the first composition of the first product portion 102 is free of dissolved elements from the second composition. The second composition of the second product portion 104 may be free of dissolved elements from the first composition. In at least one example embodiment, the first and second product portions 102, 104 adhere to one another, but are not intermixed. Thus, the first and second product portions 102, 104 may be described as being non-mixed. In at least one example embodiment, the first product portion 102 is substantially homogeneous such that it is free of a compositional gradient. In at least one example embodiment, the second product portion 104 is substantially homogeneous such that it is free of a compositional gradient.

In at least one example embodiment, each of the first product portions 102 defines a first thickness 106 and each of the second product portions 104 defines a second thickness 108. The first and second thicknesses 106, 108 may be the same or different. When the oral product 100 includes a plurality of the first product portions 102, each of the first product portions 102 may have the same thickness (i.e., the first thickness 106, as shown), or all or a portion of the first product portions 102 may have different thicknesses. When the oral product 100 includes a plurality of the second product portions 104, each of the second product portions 104 may have the same thickness (i.e., the second thickness 108, as shown), or all or a portion of the second product portions 104 may have different thicknesses.

In at least one example embodiment, the first thickness 106 is greater than or equal to about 0.5 mm (e.g., greater than or equal to about 0.6 mm, greater than or equal to about 0.7 mm, greater than or equal to about 0.8 mm, greater than or equal to about 0.9 mm, greater than or equal to about 1 mm, greater than or equal to about 1.1 mm, greater than or equal to about 1.2 mm, greater than or equal to about 1.3 mm, greater than or equal to about 1.4 mm, greater than or equal to about 1.5 mm, or greater than or equal to about 2 mm). The first thickness 106 may be less than or equal to about 2.5 mm (e.g., less than or equal to about 2 mm, less than or equal to about 1.5 mm, less than or equal to about 1.4 mm, less than or equal to about 1.3 mm, less than or equal to about 1.2 mm, less than or equal to about 1.1 mm, less than or equal to about 1 mm, less than or equal to about 0.9 mm, less than or equal to about 0.8 mm, less than or equal to about 0.7 mm, or less than or equal to about 0.6 mm). In at least one example embodiment, the second thickness 108 is greater than or equal to about 0.5 mm (e.g., greater than or equal to about 0.6 mm, greater than or equal to about 0.7 mm, greater than or equal to about 0.8 mm, greater than or equal to about 0.9 mm, greater than or equal to about 1 mm, greater than or equal to about 1.1 mm, greater than or equal to about 1.2 mm, greater than or equal to about 1.3 mm, greater than or equal to about 1.4 mm, greater than or equal to about 1.5 mm, or greater than or equal to about 2 mm). The second thickness 108 may be less than or equal to about 2.5 mm (e.g., less than or equal to about 2 mm, less than or equal to about 1.5 mm, less than or equal to about 1.4 mm, less than or equal to about 1.3 mm, less than or equal to about 1.2 mm, less than or equal to about 1.1 mm, less than or equal to about 1 mm, less than or equal to about 0.9 mm, less than or equal to about 0.8 mm, less than or equal to about 0.7 mm, or less than or equal to about 0.6 mm).

In at least one example embodiment, at least one of the first and second product portions 102, 104 includes nicotine. The other of the first and second product portions 102, 104 may include a nicotine masking agent. In at least one example embodiment, both of the first and second product portions 102, 104 include nicotine. The first composition includes nicotine in a first amount (e.g., weight) and the second composition includes nicotine in a second amount that is different than the first amount.

In at least the example embodiment shown, the first and second product portions 102, 104 are in the form of layers. The first and second product portions 102, 104 are in alternating layers. A quantity of first product portions 102 may be the same as or different than a quantity of second product portions 104. The oral product 100 may have a total quantity of portions or layers greater than or equal to about 2 (e.g., greater than or equal to about 3, greater than or equal to about 4, greater than or equal to about 5, greater than or equal to about 6, greater than or equal to about 7, greater than or equal to about 8, or greater than or equal to about 9). The total quantity of layers may be less than or equal to about 10 (e.g., less than or equal to about 9, less than or equal to about 8, less than or equal to about 7, less than or equal to about 6, less than or equal to about 5, less than or equal to about 4, or less than or equal to about 3).

The oral product 100 includes a first or base layer 110. The base layer 110 may be the first layer that is printed on a build surface during manufacturing of the oral product 110. In at least the example embodiment shown, the base layer 110 is one of the first product portions 102. In the example embodiment shown, the base layer 110 defines a third thickness 112 that is the same as the first thickness 106 of a remainder of the first product portions 102. In at least one other example embodiment, a third thickness of a base layer is different from (e.g., greater than or less than) a thickness of other layers.

FIG. 2 is a perspective view of another oral product according to at least one example embodiment.

In at least one example embodiment, an oral product 200 includes a first product portion 202, a second product portion 204, a third product portion 206, and a fourth product portion 208. Except for quantity, arrangement, and size of the product portions 202, 204, 206, 208, the oral product 200 is the same as the oral product 100 (shown in FIG. 1). The product portions 202, 204, 206, 208 cooperate to form a grid pattern.

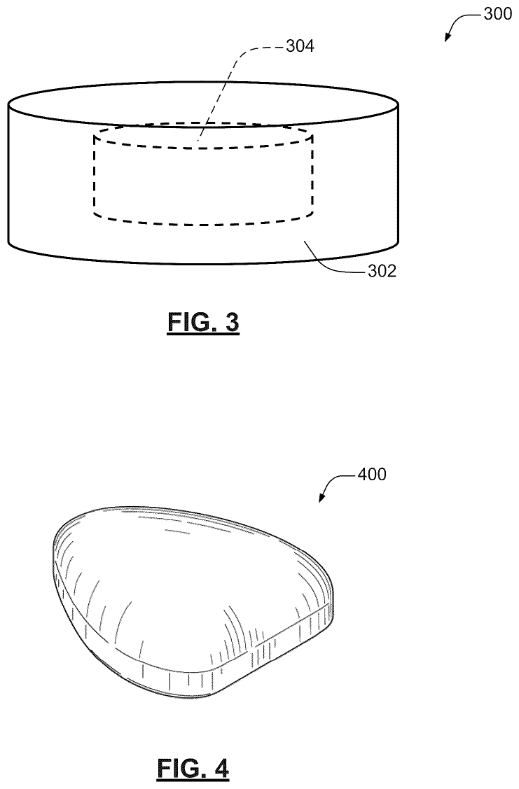

FIG. 3 is a perspective view of another oral product according to at least one example embodiment.

In at least one example embodiment, an oral product 300 includes a first product portion 302 and a second product portion 304. Except for shape, size, and arrangement of the first and second product portions 302, 304, the oral product 300 is the same as the oral product 100 (shown in FIG. 1). In at least one example embodiment, the first product portion 302 surrounds the second product portion 304.

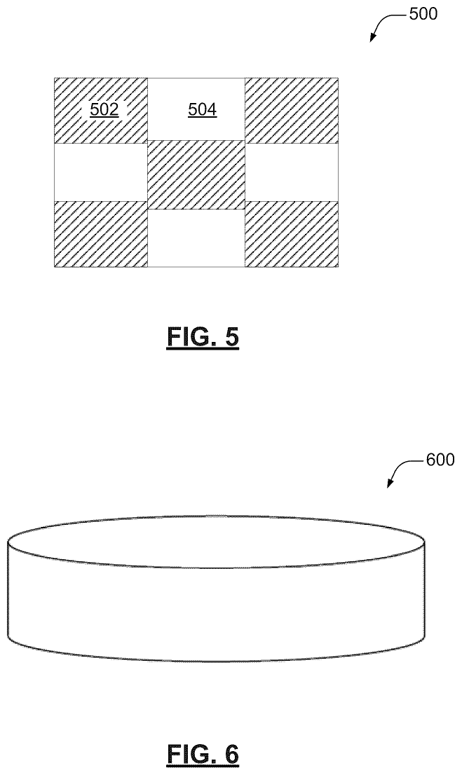

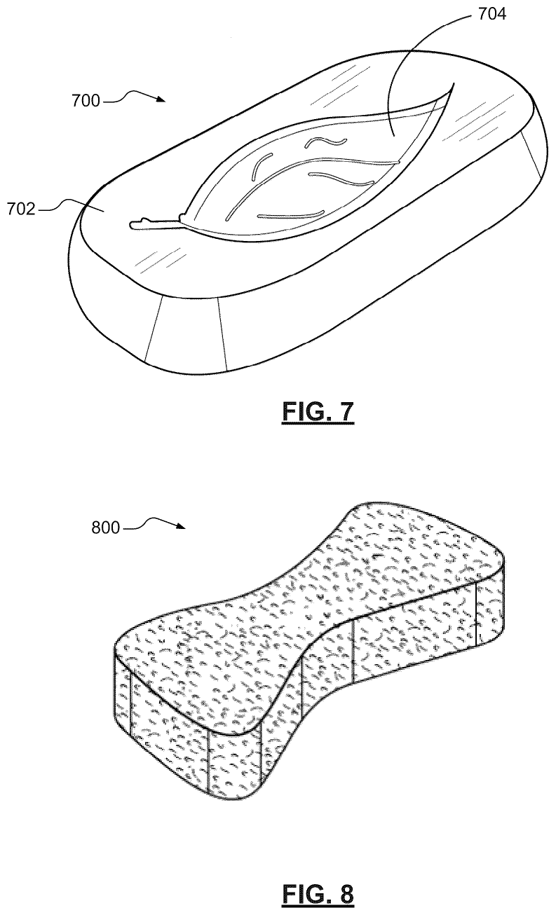

The oral products 100, 200, 300 of FIGS. 1-3, respectively, may have any format that is compatible with three-dimensional printing. For example, the oral product may be a lozenge (FIG. 4), a film (FIG. 5), a chewing gum (FIG. 6), a dissolvable tablet (FIG. 7), and/or a non-dissolvable chew (FIG. 8), as discussed in greater detail below.

In at least one example embodiment, the oral product (e.g., oral products 100, 200, 300) is a lozenge. In at least one example embodiment, the lozenge may be a lozenge as described in U.S. Pat. No. 9,351,936 as issued May 31, 2016 and titled NICOTINE LOZENGE and/or U.S. Pub. No. 2022/0312825 as filed Apr. 6, 2021 and titled ORAL POUCH PRODUCT, the entire contents of which are hereby incorporated by reference. When the lozenge includes two or more product portions, each of the product portions may independently have a composition as described in the above patent documents.

FIG. 4 is a perspective view of an example lozenge according to at least one example embodiment.

By way of example, FIG. 4 is a perspective view of a lozenge 400 according to at least one example embodiment. As illustrated, in at least one example embodiment, the lozenge 400 may have a rounded shield shape. However, it should be appreciated that the lozenge 400 may have a variety of other shapes and/or sizes and/or configurations, as detailed above. In at least one example embodiment, the lozenge 400 may include two or more product portions or regions, as described above in the discussion accompanying FIGS. 1-3.

In at least one example embodiment, the lozenge 400 may include, for example, a solid solution and a liquid mixture. In at least one example embodiment, the lozenge includes two or more product portions, with one or more of the product portions including a solid solution and a liquid mixture. The solid solution may include, for example, soluble fibers and a sugar alcohol, such as those detailed above. For example, in at least one example embodiment, the solid solution includes isomalt. In at least one example embodiment, the liquid mixture is absorbed in a carrier. In at least one example embodiment, the lozenges may directly include the liquid mixture without a carrier. The liquid mixture may be configured to increase buccal nicotine absorption. In at least one example embodiment, the liquid mixture may include, for example, nicotine and a triglyceride. The nicotine may include a liquid nicotine and at least a part of the liquid nicotine may be dissolved in the triglyceride. The triglyceride may include, for example, medium-chain triglyceride (MCT).

In at least one example embodiment, the oral product is a dissolvable film. The dissolvable film may be a film as described, for example, in U.S. Pat. No. 8,469,036 as issued Jun. 25, 2013 and titled TOBACCO COMPOSITIONS, the entire contents of which is hereby incorporated by reference. When the dissolvable film includes two or more product portions, each of the product portions may independently have a composition as described in the above patent document.

FIG. 5 is a top-down view of an example dissolvable film according to at least one example embodiment.

By way of example, FIG. 5 is a top-down view of a dissolvable film 500 according to at least one example embodiment. As illustrated, in at least one example embodiment, the dissolvable film 500 includes a pattern defined by two or more first regions 502 and two or more second regions 504.

In at least one example embodiment, the dissolvable film 500 may include one or more continuous or discontinuous layers. The one or more continuous or discontinuous layers may each have the same or different pattern. The one or more layers, discontinuous or continuous and have the same or different patterns, may together define the oral product, and the oral product may have an overall thickness, for example, of less than or equal to about 1 mm (e.g., less than or equal than or equal to about 500 micrometers (μm), less than or equal to about 200 μm, less than or equal to about 100 μm, less than or equal to about 50 μm, less than or equal to about 40 μm, less than or equal to about 30 μm, less than or equal to about 20 μm, less than or equal to about 10 μm, less than or equal to about 5 μm, less than or equal to about 4 μm, less than or equal to about 3 μm, less than or equal to about 2 μm, or less than or equal to about 1 μm). In at least one example embodiment, the dissolvable film 500 may have a thickness ranging from about 5 μm to about 125 μm.

In at least one example embodiment, the oral product is a chewing gum. The chewing gum may be a chewing gum as described, for example, in U.S. Pub. No. 2022/0313679 as filed Apr. 6, 2021 and titled CONTROLLED-RELEASE NICOTINE CHEWING GUM, the entire contents of which is hereby incorporated by reference. When the chewing gum includes two or more product portions, each of the product portions may independently have a composition as described in the above patent document.

FIG. 6 is a perspective view of an example chewing gum according to at least one example embodiment.

By way of example, FIG. 6 is a perspective view of a chewing gum 600 according to at least one example embodiment. As illustrated, in at least one example embodiment, the chewing gum 600 may have a substantially oval-shaped cross section. However, it should be appreciated that the chewing gum 600 may have a variety of other shapes and/or sizes and/or configurations, as detailed above. In at least one example embodiment, the chewing gum 600 includes two or more portions or regions, as described above in the discussion accompanying FIGS. 1-3.

In at least one example embodiment, the chewing gum 600 includes a gum base polymer, an oil, and a nicotine or a nicotine derivative. In at least one example embodiment, the chewing gum 600 includes two or more product portions, with one or more of the product portions including a gum base polymer, an oil, and a nicotine or a nicotine derivative. The gum base polymer may include, for example, polyvinyl acetate (PVA). The oil may include, for example, triglyceride.

In at least one example embodiment, the oral product is a dissolvable tablet. The dissolvable tablet may be a dissolvable tablet as described, for example, in U.S. Pat. No. 11,771,127 as issued Oct. 3, 2023 and titled CHEWABLE DISSOLVABLE NICOTINE TABLET and/or U.S. Pat. No. 9,930,909 as issued Apr. 3, 2018 and titled ORAL PRODUCT and/or U.S. Pat. No. 8,469,036 as issued Jun. 25, 2013 and titled TOBACCO COMPOSITIONS, the entire contents of each are hereby incorporated by reference. When the dissolvable tablet includes two or more product portions, each of the product portions may independently have a composition as described in the above patent documents.

FIG. 7 is a perspective view of an example dissolvable tablet according to at least one example embodiment.

By way of example, FIG. 7 is a perspective view of a dissolvable tablet 700 according to at least one example embodiment. In at least one example embodiment, the dissolvable tablet 700 may include two or more product portions, as described above in the discussion accompanying FIGS. 1-3. As illustrated, in at least one example embodiment, the dissolvable table 700 may have a rounded-edge rectangular shape. However, it should be appreciated that the dissolvable tablet 700 may have a variety of other shapes and/or sizes and/or configurations, as detailed above.

As illustrated, in at least one example embodiment, a first surface 702 of the dissolvable tablet 700 may be embossed or stamped with any type of indicia 704. However, it should be appreciated that, in at least one example embodiment, the dissolvable tablet 700 is free of any indicia.

In at least one example embodiment, the oral product is a non-dissolvable chew. The non-dissolvable chew may be a non-dissolvable chew as described, for example, in U.S. Pat. No. 9,854,831 as issued Jan. 2, 2018 and titled ORAL PRODUCT and/or U.S. Pat. No. 10,098,376 as issued Oct. 16, 2018 and titled TOBACCO COMPOSITIONS and/or U.S. Pat. No. 9,420,827 as issued Aug. 23, 2016 and titled SOFT ORAL PRODUCT and/or U.S. Pat. No. 9,185,931 as issued Nov. 17, 2015 and titled ORAL PRODUCT, the entire contents of each are hereby incorporated by reference. When the non-dissolvable chew includes two or more product portions, each of the product portions may independently have a composition as described in the above patent documents.

FIG. 8 is a perspective view of an example non-dissolvable chew according to at least one example embodiment.

By way of example, FIG. 8 is a perspective view of a non-dissolvable chew 800 according to at least one example embodiment. In at least one example embodiment, the non-dissolvable chew 800 includes two or more product portions, as described above in the discussion accompanying FIGS. 1-3. As illustrated, in at least one example embodiment, the non-dissolvable chew 800 may have a bowtie shape. However, it should be appreciated that the non-dissolvable chew 800 may have a variety of other shapes and/or sizes and/or configurations, as detailed above.

The present disclosure provides methods for forming oral products like those detailed above.

In at least one example embodiment, the present disclosure provides three-dimensional printing methods for forming the oral products.

In at least one example embodiment, the method includes disposing a first composition using a three-dimensional printer to form one or more first portions of the oral product and disposing a second composition using the three-dimensional printer to form one or more second portions of the oral products. In at least one example embodiment, the first and second portions may be disposed simultaneously. In at least one example embodiment, the first and second portions may be disposed concurrently. In at least one example embodiment, the first and second portions may be disposed subsequently.

In at least one example embodiments, the three-dimensional printer may be configured to form the one or more first portions by disposing the first composition in one or more distinct first layers. In at least one example embodiments, the three-dimensional printer may be configured to form the one or more second portions by disposing the second composition in one or more distinct second layers. In at least one example embodiment, the three-dimensional printer may be configured to disposed the one or more first layers around the one or more second layers. In at least one example embodiment, the three-dimensional printer may be configured to disposed the one or more second layers around the one or more first layers. In at least one example embodiment, the three-dimensional printer may be configured to disposed the one or more first layers between the one or more second layers. In at least one example embodiment, the three-dimensional printer may be configured to disposed the one or more second layers between the one or more first layers.

In at least one example embodiment, the three-dimensional printer may be configured to dispose the first composition at a first rate and the second composition at a second rate. In at least one example embodiment, the second rate may be different from the first rate.

In at least one example embodiments, the three-dimensional printer may include a part or component or portion configured to receive, hold, and dispose the first composition and the second composition.

In at least one example embodiments, the three-dimensional printer may include a first part or component or portion configured to receive, hold, and dispose the first composition and a second part or component or portion distinct from the first part that is configured to receive, hold, and dispose the second composition.

The first and second compositions may be the same or different. For example, in at least one example embodiment, the first and second compositions may include the same or different compounds or elements or components. The first and second compositions may include, for example, any one or combination of the compounds or elements or components as detailed above. For example, in at least one example embodiment, the first composition includes a tobacco extract, such as a tobacco-derived nicotine extract and/or synthetic nicotine. The first composition may include the tobacco-derived nicotine extract and/or synthetic nicotine alone or in combination with a carrier. In at least one example embodiment, the first composition includes, additionally, or alternatively, nicotine or a nicotine derivative. For example, the first composition may include a complex of nicotine or a salt of nicotine. In at least one example embodiment, the first composition includes, additionally, or alternatively, cannabis, such as cannabis plant tissue and/or cannabis extracts. In at least one example embodiment, the first composition includes, additionally, or alternatively, one or more compounds or elements or components such as a mouth-stable polymer, a mouth-soluble polymer, a sweetener, an energizing agent, a soothing agent, a focusing agent, a plasticizer, mouth-soluble fibers, an alkaloid, a mineral, a vitamin, a dietary supplement, a nutraceutical, a coloring agent, an amino acid, a chemesthetic agent, an antioxidant, a food-grade emulsifier, a pH modifier, a botanical, a tooth-whitening agent, a therapeutic agent, a processing aid, a stearate, a wax, a stabilizer, a disintegrating agent, a lubricant, a preservative, a filler, a flavorant, flavor masking agents, a bitterness receptor site blocker, a receptor site enhancers, a carrier, an oil, a base polymer, one or more additives, or any combination thereof.

Similarly, in at least one example embodiment, the second composition includes a tobacco extract, such as a tobacco-derived nicotine extract and/or synthetic nicotine. The second composition may include the tobacco-derived nicotine extract and/or synthetic nicotine alone or in combination with a carrier. In at least one example embodiment, the second composition includes, additionally, or alternatively, nicotine or a nicotine derivative. For example, the second composition may include a complex of nicotine or a salt of nicotine. In at least one example embodiment, the second composition includes, additionally, or alternatively, cannabis, such as cannabis plant tissue and/or cannabis extracts. In at least one example embodiment, the second composition includes, additionally, or alternatively, one or more compounds or elements or components such as a mouth-stable polymer, a mouth-soluble polymer, a sweetener, an energizing agent, a soothing agent, a focusing agent, a plasticizer, mouth-soluble fibers, an alkaloid, a mineral, a vitamin, a dietary supplement, a nutraceutical, a coloring agent, an amino acid, a chemesthetic agent, an antioxidant, a food-grade emulsifier, a pH modifier, a botanical, a tooth-whitening agent, a therapeutic agent, a processing aid, a stearate, a wax, a stabilizer, a disintegrating agent, a lubricant, a preservative, a filler, a flavorant, flavor masking agents, a bitterness receptor site blocker, a receptor site enhancers, a carrier, an oil, a base polymer, a masking agent, one or more additives, or any combination thereof.

In at least one example embodiments, the first and second compositions may include the same or different densities. The first composition may have a first density, and the second composition may have a second density. In at least one example embodiment, the second density may be different from the first density.

In at least one example embodiment, the first composition may include a first nicotine amount, and the second composition may include a second nicotine amount. In at least one example embodiment, the second nicotine amount may be different from the first nicotine amount.

In at least one example embodiment, the first composition may include a first flavorant amount, and the second composition may include a second flavorant amount. In at least one example embodiment, the second flavorant amount may be different from the first flavorant amount.

In at least one example embodiment, the first composition may include a first flavorant, and the second composition may include a second flavorant. In at least one example embodiment, the second flavorant may be different than the first flavorant.

In at least one example embodiment, the oral product may include a first amount of the first composition and a second amount of the second composition. In at least one example embodiment, the first amount of the first composition may be greater than the second amount of the second composition—i.e., the oral product includes more of the first composition than the second composition.

In at least one example embodiment, the method may include, in addition to disposing the first composition and the second composition, disposing a third composition using the three-dimensional printer to form one or more third portions of the oral product. In at least one example embodiment, the first, second, and third portions may be disposed simultaneously. In at least one example embodiment, the first, second, and third portions may be disposed concurrently. In at least one example embodiment, the first, second, and third portions may be disposed subsequently. In at least one example embodiment, the first and second portions may be disposed simultaneously or concurrently, and the third portion may be disposed subsequently. In at least one example embodiment, the first and third portions may be disposed simultaneously or concurrently, and the second portion may be disposed subsequently. In at least one example embodiment, the second and third portions may be disposed simultaneously or concurrently, and the first portion may be disposed subsequently.

In at least one example embodiment, the three-dimensional printer may be configured to form the one or more third portions by disposing the third composition in one or more distinct third layers. In at least one example embodiment, the three-dimensional printer may be configured to disposed the one or more third layers around the one or more first layers and/or the one or more second layers. In at least one example embodiment, the three-dimensional printer may be configured to disposed the one or more first layers and/or the one or more second layers around the one or more third layers. In at least one example embodiment, the three-dimensional printer may be configured to disposed the one or more third layers between the one or more first layers and/or the one or more second layers. In at least one example embodiment, the three-dimensional printer may be configured to disposed the one or more first layers and/or the one or more second layers between the one or more third layers.

In at least one example embodiment, the three-dimensional printer may be configured to dispose the third composition at a third rate. In at least one example embodiment, the third rate may be different from the first rate and/or the second rate.

In at least one example embodiments, the three-dimensional printer may include a part or component or portion configured to receive, hold, and dispose the first composition and the second composition and/or the third composition.

In at least one example embodiments, the three-dimensional printer may include a part or component or portion configured to receive, hold, and dispose the second composition and the first composition and/or third composition.

In at least one example embodiments, the three-dimensional printer may include a third part or component or portion distinct from the first part or component or portion and/or the second part or component or portion configured to receive, hold, and dispose the third composition.

In at least one example embodiment, the third composition may be the same or different from the first composition and/or the second composition. For example, in at least one example embodiment, the third composition may be the same as the first composition. In at least one example embodiment, the third composition may be the same as the second composition. In at least one example embodiment, the third composition may be the different from the first composition and the second composition.

The third composition may include, for example, any one or combination of the compounds or elements or components as detailed above. For example, in at least one example embodiment, the first composition includes a tobacco extract, such as a tobacco-derived nicotine extract and/or synthetic nicotine. The first composition may include the tobacco-derived nicotine extract and/or synthetic nicotine alone or in combination with a carrier. In at least one example embodiment, the first composition includes, additionally, or alternatively, nicotine or a nicotine derivative. For example, the first composition may include a complex of nicotine or a salt of nicotine. In at least one example embodiment, the first composition includes, additionally, or alternatively, cannabis, such as cannabis plant tissue and/or cannabis extracts. In at least one example embodiment, the first composition includes, additionally, or alternatively, one or more compounds or elements or components such as a mouth-stable polymer, a mouth-soluble polymer, a sweetener, an energizing agent, a soothing agent, a focusing agent, a plasticizer, mouth-soluble fibers, an alkaloid, a mineral, a vitamin, a dietary supplement, a nutraceutical, a coloring agent, an amino acid, a chemesthetic agent, an antioxidant, a food-grade emulsifier, a pH modifier, a botanical, a tooth-whitening agent, a therapeutic agent, a processing aid, a stearate, a wax, a stabilizer, a disintegrating agent, a lubricant, a preservative, a filler, a flavorant, flavor masking agents, a bitterness receptor site blocker, a receptor site enhancers, a carrier, an oil, a base polymer, one or more additives, or any combination thereof.

In at least one example embodiments, the first, second, and third compositions may include the same or different densities. The first composition may have a first density, the second composition may have a second density, and the third composition may have a third density. In at least one example embodiment, the third density may be different from the first density and/or the second density.

In at least one example embodiment, the first composition may include a first nicotine amount, the second composition may include a second nicotine amount, and the third composition may include a third nicotine amount. In at least one example embodiment, the third nicotine amount may be different from the first nicotine amount and/or the second nicotine amount.

In at least one example embodiment, the first composition may include a first flavorant amount, the second composition may include a second flavorant amount, and the third flavorant amount. In at least one example embodiment, the third flavorant amount may be different from the first flavorant amount and/or the second flavorant.

In at least one example embodiment, the first composition may include a first flavorant, the second composition may include a second flavorant, and the third composition may include a third flavorant. In at least one example embodiment, the third flavorant may be different than the first flavorant and/or the second flavorant.

In at least one example embodiment, the first composition may include nicotine, the second composition may include a masking agent, and the third composition may include a flavorant. For example, in at least one example embodiment, the first composition may include a first nicotine amount, the second composition may include a second nicotine amount that is less than the first nicotine amount, and the third composition may include a third nicotine amount that is also less than the first nicotine amount. In at least one example embodiment, the second composition may include a first masking agent amount, the first composition may include a second masking agent amount that is less than the first masking agent amount, and the third composition may include a third masking agent amount that is also less than the first masking agent amount. In at least one example embodiment, the third composition may include a first flavorant amount, the first composition may include a second flavorant amount that is less than the first flavorant amount, and the second composition may include a third flavorant amount that is also less than the first flavorant amount.

In at least one example embodiment, the oral product may include a third amount of the third composition. In at least one example embodiment, the third amount of the third composition may be less than the second amount of the second composition and/or the first amount of the first composition—i.e., the oral product includes more of the first composition than the third composition and/or the oral product includes more of the second composition than the third composition.

Although three compositions are described, it should be appreciated that in various embodiments, the method may include disposing any number of same or different compositions to define one or more parts of the oral product.

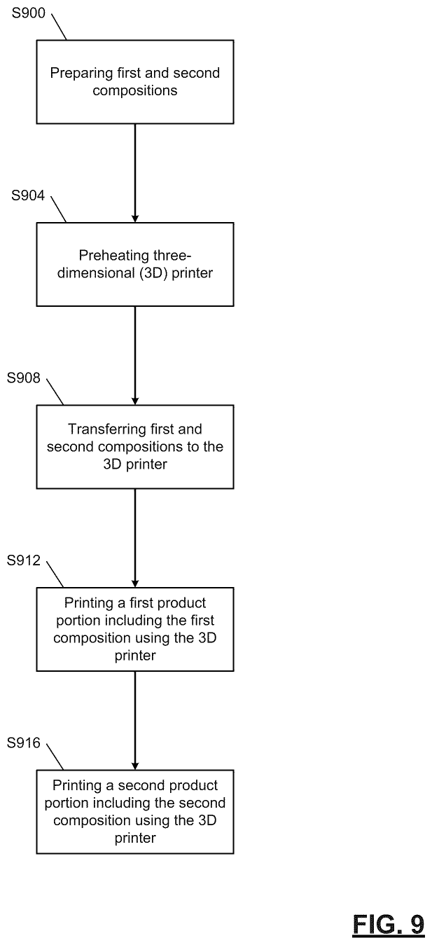

FIG. 9 is a flow diagram illustrating a method of making an oral product according to at least one example embodiment.

As shown in FIG. 9, a method of making an oral product (e.g., the oral product of any of FIGS. 1-8) generally includes preparing first and second compositions at S900; preheating a 3D printer at S904; transferring first and second compositions to the 3D printer at S908; printing a first product portion including the first composition using the 3D printer at S912; and printing a second product portion including the second composition using the 3D printer at S916. The method may include repeating steps S912 and S916 to create a desired quantity of first and second product portions. Each of these steps is discussed in greater detail below.

At S900, the method includes preparing first and second compositions. In at least one example embodiment, S900 includes preparing compositions for a desired product format. The first and second compositions may be in the form of solutions and/or slurries. In at least one example embodiment, each of the solutions and/or slurries includes a solvent, such as water. S900 may include removing at least a portion of the solvent, such as greater than or equal to about 75 weight percent of the solvent (e.g., greater than or equal to about 80 weight percent, greater than or equal to about 85 weight percent, greater than or equal to about 90 weight percent, greater than or equal to about 92 weight percent, greater than or equal to about 95 weight percent, greater than or equal to about 97 weight percent, greater than or equal to about 98 weight percent, or greater than or equal to about 99 weight percent). In at least one example embodiment, S900 includes stirring each of the solution and/or slurry.

In at least one example embodiment, preparing the first composition includes heating a first solution and/or slurry to a first desired (or alternatively, predetermined) temperature. The first desired temperature may be greater than or equal to about 40° C. (e.g., greater than or equal to about 50° C., greater than or equal to about 60° C., greater than or equal to about 70° C., greater than or equal to about 80° C., greater than or equal to about 90° C., greater than or equal to about 100° C., greater than or equal to about 110° C., greater than or equal to about 120° C., greater than or equal to about 130° C., greater than or equal to 140° C., greater than or equal to about 150° C., greater than or equal to about 160° C., greater than or equal to about 170° C., greater than or equal to about 180° C., greater than or equal to about 190° C., greater than or equal to about 200° C., greater than or equal to about 210° C., greater than or equal to about 220° C., greater than or equal to about 230° C., greater than or equal to about 240° C., or greater than or equal to about 250° C.). The first desired temperature may be less than or equal to about 280° C. (e.g., less than or equal to about 250° C., less than or equal to about 240° C., less than or equal to about 230° C., less than or equal to about 220° C., less than or equal to about 210° C., less than or equal to about 200° C., less than or equal to about 190° C., less than or equal to about 80° C., less than or equal to about 170° C., less than or equal to about 160° C., less than or equal to about 150° C., less than or equal to about 140° C., less than or equal to about 130° C., less than or equal to about 120° C., less than or equal to about 110° C., less than or equal to about 100° C., less than or equal to about 90° C., less than or equal to about 80° C., less than or equal to about 70° C., less than or equal to about 60° C., or less than or equal to about 50° C.).

Preparing the second composition may include heating a second solution and/or slurry to a second desired (or alternatively, predetermined) temperature. The above ranges for the first desired temperature are also applicable to the second desired temperature. The second desired temperature may be the same as the first desired temperature or different from the first desired temperature.

In at least one example embodiment, the first and second compositions are lozenge precursor compositions. The first desired temperature is higher than a first crystallization point of the first composition. The second desired temperature is higher than a second crystallization point of the second composition. In at least one example embodiment, the first desired temperature is greater than or equal to about 150° C. (e.g., greater than or equal to about 151° C., greater than or equal to about 152° C., greater than or equal to about 153° C., greater than or equal to about 154° C., greater than or equal to about 155° C., greater than or equal to about 156° C., greater than or equal to about 157° C., greater than or equal to about 158° C., or greater than or equal to about 159° C.). The first desired temperature may be less than or equal to about 160° C. (e.g., less than or equal to about 159° C., less than or equal to about 158° C., less than or equal to about 157° C., less than or equal to about 156° C., less than or equal to about 155° C., less than or equal to about 154° C., less than or equal to about 153° C., less than or equal to about 152° C., or less than or equal to about 151° C.). The above ranges for the first desired temperature are also applicable to the second desired temperature. The second desired temperature may be the same as the first desired temperature or different from the first desired temperature.

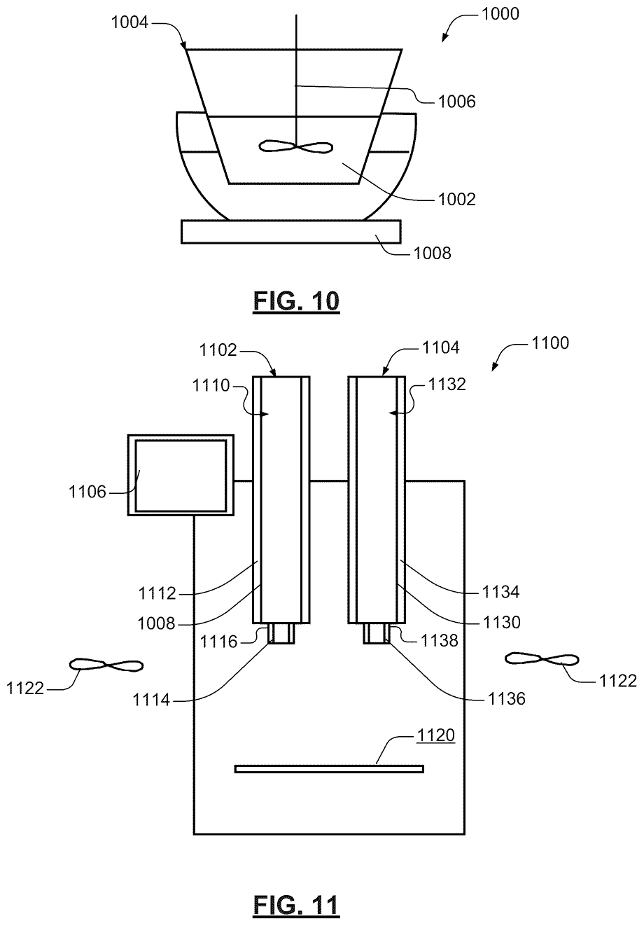

FIG. 10 is a schematic illustration of equipment preparing a composition according to at least one example embodiment.

In at least one example embodiment, FIG. 10 illustrates example composition preparation equipment 1000. The method may include preparing a solution and/or slurry 1002 (i.e., a composition precursor) in a double boiler 1004. The solution and/or slurry 1002 may be mixed and/or stirred using a stirrer 1006. The solution and/or slurry 1002 may be heated, such as by a hot plate 1008. The first and second compositions may each be or include liquids.

Returning to FIG. 9, at S904, the method includes preheating a 3D printer. S904 may include preheating a first component or part (e.g., a first syringe and/or nozzle) of the 3D printer to a third desired (or alternatively, predetermined) temperature. The first component is configured to hold and dispense the first composition. In at least one example embodiment, the third desired temperature is less than the first desired temperature. The third desired temperature may be greater than or equal to about 65° C. (e.g., greater than or equal to about 67.5° C., greater than or equal to about 70° C., greater than or equal to about 72.5° C., greater than or equal to about 75° C., or greater than or equal to about 77.5° C.). The third desired temperature may be less than or equal to about 80° C. (e.g., less than or equal to about 77.5° C., less than or equal to about 75° C., less than or equal to about 72.5° C., less than or equal to about 70° C., or less than or equal to about 67.5° C.).

S904 may further include preheating a second component or part (e.g., a second syringe and/or nozzle) of the 3D printer to a fourth desired (or alternatively, predetermined) temperature. The second component is configured to hold and dispense the second composition. In at least one example embodiment, the fourth desired temperature is less than the second desired temperature. The above ranges for the third desired temperature are also applicable to the fourth desired temperature. The fourth desired temperature may be the same as the third desired temperature or different from the third desired temperature.

In at least one example embodiment, the third and fourth desired temperatures are high enough to prevent solidification of the first and second compositions, respectively. In at least one example embodiment, the third and fourth temperatures are low enough to reduce or prevent unintended discharged of the first and second compositions from the first and second components. S904 may be performed before, after, or concurrently with S900.

At S908, the method includes transferring first and second compositions to the 3D printer at S908. S908 is performed after S900 and S904. S908 includes transferring the first composition to the first component. S908 further includes transferring the second composition to the second component.

At S912, the method includes printing a first product portion including the first composition using the 3D printer. As discussed above, the first composition may be or include a liquid. The first composition may be discharged from the first component as a liquid and may cool and/or harden into a solid. In at least one example embodiment, the first composition is extruded from the first component. S900 may be performed a desired print head speed, discharge rate (i.e., rate at which the first composition is discharged from the first component), and/or temperature (discussed in greater detail below), depending, at least in part, on the characteristics of the material and the desired thickness of the product portion to be printed.

S912 may be performed at a desired environmental temperature (i.e., an environment in which the 3D printer is disposed). In at least one example embodiment, the first desired environmental temperature is greater than or equal to about 15° C. (e.g., greater than or equal to about 17.5° C., greater than or equal to about 20° C., or greater than or equal to about 22.5° C.). The desired environmental temperature may be less than or equal to about 25° C. (e.g., less than or equal to about 22.5° C., less than or equal to about 20° C., or less than or equal to about 17.5° C.).

S912 may be performed at a desired humidity (i.e., in the environment surrounding the 3D printer). The desired humidity may be greater than or equal to about 30% (e.g., greater than or equal to about 35% greater than or equal to about 40%, greater than or equal to about 45%, greater than or equal to about 50%, greater than or equal to about 55%, greater than or equal to about 60%, or greater than or equal to about 65%). The desired humidity may be less than or equal to about 70% (e.g., less than or equal to about 65%, less than or equal to about 60%, less than or equal to about 55%, less than or equal to about 50%, less than or equal to about 45%, less than or equal to about 40%, or less than or equal to about 35%).

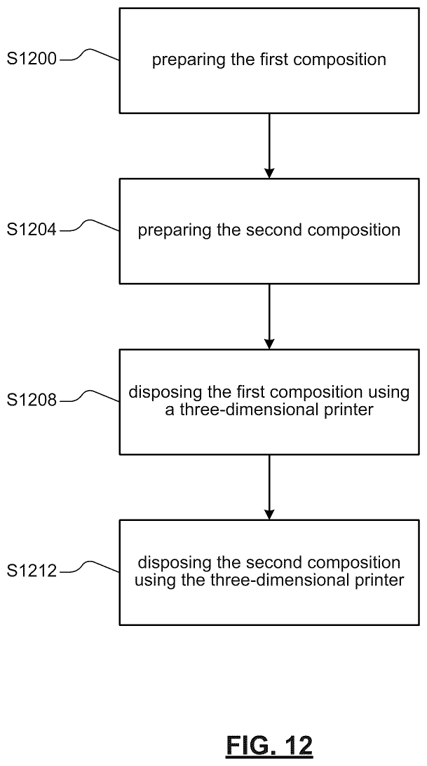

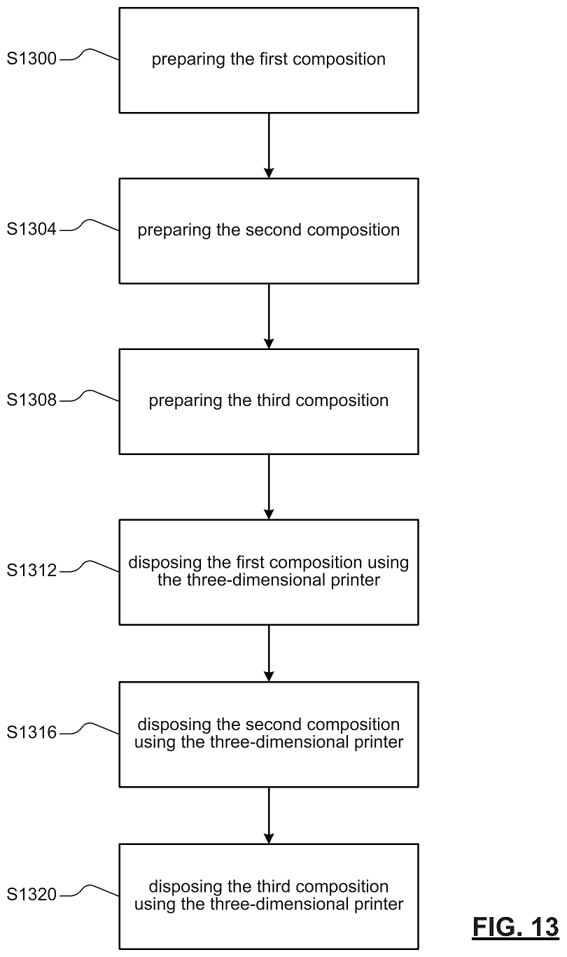

FIG. 11 is a schematic illustration of a three-dimensional printer according to at least one example embodiment.

In at least one example embodiment, S912 is performed using a 3D printer 1100. The 3D printer 1100 includes a first component or part 1102 and a second component or part 1104. The first component 1102 is configured to hold, heat, and dispense the first composition. The second component 1104 is configured to hold, heat, and dispense the second composition. The 3D printer 1100 may further include a controller (not shown) and a user interface device or display 1106.

The first component 1102 may include a first syringe 1108 defining a first interior region 1110. The first syringe 1108 may be configured to be heated by a first heater 1112, such as an external surface heater. The first component 1102 may further include a first nozzle 1114. The nozzle 1114 may be downstream of the syringe 1108. The first nozzle 1114 may be configured to be heated by a second heater 1116.

The first heater 1112 may be configured to heat the first composition in the first interior region 1110 to a fifth desired (or alternatively, predetermined) temperature. The fifth desired temperature may be greater than the third desired temperature. The fifth desired temperature may be less than the first desired temperature. The fifth desired temperature may be low enough to reduce or prevent unintended discharge of the first composition from the first component 1102. In at least one example embodiment, the fifth desired temperature is greater than or equal to about 80° C. (e.g., greater than or equal to about 81° C., greater than or equal to about 82° C., greater than or equal to about 83° C., or greater than or equal to about 84° C.). The fifth desired temperature may be less than or equal to about 85° C. (e.g., less than or equal to about 84° C., less than or equal to about 83° C., less than or equal to about 82° C., or less than or equal to about 81° C.).

The second heater 1116 may be configured to heat the first composition in the first nozzle 1114 to a sixth desired (or alternatively, predetermined) temperature. The sixth desired temperature may be greater than the fifth desired temperature. The sixth desired temperature may be greater than the third desired temperature. The sixth desired temperature may be less than the first desired temperature. In at least one example embodiment, the sixth desired temperature is greater than or equal to about 90° C. (e.g., greater than or equal to about 91° C., greater than or equal to about 92° C., greater than or equal to about 93° C., greater than or equal to about 94° C., greater than or equal to about 95° C., greater than or equal to about 96° C., greater than or equal to about 97° C., greater than or equal to about 98° C., greater than or equal to about 99° C., greater than or equal to about 100° C., greater than or equal to about 101° C., greater than or equal to about 102° C., greater than or equal to about 103° C., or greater than or equal to about 104° C.). The sixth desired temperature may be less than or equal to about 105° C. (e.g., less than or equal to about 104° C., less than or equal to about 103° C., less than or equal to about 102° C., or less than or equal to about 101° C., less than or equal to about 100° C., less than or equal to about 99° C., less than or equal to about 98° C., or less than or equal to about 97° C., less than or equal to about 96° C., less than or equal to about 94° C., less than or equal to about 93° C., or less than or equal to about 92° C., or less than or equal to about 91° C.).

S912 may include printing a base product portion on a build surface 1120 (i.e., a first product portion). In at least one example embodiment, the build surface 1120 is unheated and is approximately at the desired environmental temperature. In at least one other example embodiment, the build surface 1120 is cooled below the desired environmental temperature. S912 may include circulating air in an environment 1122 surrounding the build surface 1120, such as by one or more fans 1124. Circulating air may facilitate quicker cooling and/or solidification of the first product portion.

At S916, the method includes printing a second product portion including the second composition using the 3D printer. In at least one example embodiment, S916 is performed after S912 such that the first product portion printed in S912 is completely solidified and/or cooled prior to beginning S916. Completely solidifying the first product portion prior to beginning S916 may facilitate the formation of distinct, non-mixed product regions. S916 may be performed immediately after S916. In at least one example embodiment, the first product portion may solidify and/or cool during the extrusion/printing process. In at least one other example embodiment, the method may include pausing printing for a desired (or alternatively, predetermined) amount of time between S912 and S916.

In at least one example embodiment, S916 is performed using the 3D printer 1100. The second component 1104 may include a second syringe 1130 defining a second interior region 1132. The second syringe 1130 may be configured to be heated by a third heater 1134, such as an external surface heater. The second component 1104 may further include a second nozzle 1136. The second nozzle 1136 may be downstream of the second syringe 1130. The second nozzle 1136 may be configured to be heated by a fourth heater 1138.

The third heater 1134 may be configured to heat the second composition in the second interior region 1132 to a seventh desired (or alternatively, predetermined) temperature. The seventh desired temperature may be greater than the fourth desired temperature. The seventh desired temperature may be less than the second desired temperature. The seventh desired temperature may be low enough to reduce or prevent unintended discharge of the second composition from the second component 1104. The above ranges for the fifth desired temperature are also applicable to the seventh desired temperature. The seventh desired temperature may be the same as the fifth desired temperature or different from the fifth desired temperature.

The fourth heater 1138 may be configured to heat the second composition in the second nozzle 1136 to an eighth desired (or alternatively, predetermined) temperature. The eighth desired temperature may be greater than the sixth desired temperature. The eighth desired temperature may be greater than the fourth desired temperature. The eighth desired temperature may be less than the second desired temperature. The above ranges for the sixth desired temperature are also applicable to the eighth desired temperature. The eighth desired temperature may be the same as the sixth desired temperature or different from the sixth desired temperature.

S916 may include printing the second product portion directly on the first product portion. S916 may further include circulating air in the environment 1122 surrounding the build surface 1120 to facilitate quicker cooling and/or solidification of the second product portion.

The method may further include repeating S912 and/or S916 as desired to form a desired quantity of first and second product portions. As discussed in greater detail below, the method may further include printing different or additional product portions.

FIG. 12 is a flow diagram illustrating another method for preparing an oral product according to at least one example embodiment.

By way of example, FIG. 12 is a flow diagram illustrating an example method for preparing an oral product (including, for example, any of the oral products of FIGS. 1-8) according to at least one example embodiment.

As illustrated, in at least one example embodiment, the method includes disposing S1100 a first composition using a three-dimensional printer and disposing S1204 a second composition using the three-dimensional printer. As described above, the second composition may be the same as or different from the first composition, and the three-dimensional printer may include a first part or component or portion configured to receive, hold, and dispose the first composition and a second part or component or portion distinct from the first part that is configured to receive, hold, and dispose the second composition. Although the disposing S1204 of the second composition is illustrated as following the disposing S1200 of the first composition, it should be appreciated, as described above, that the disposing S1200 of the first composition and the disposing S1204 of the second composition may occur simultaneously, concurrently, or subsequently.

In at least one example embodiment, the method may further include preparing S1108 the first composition and/or preparing S1212 the second composition. In at least one example embodiment, the preparing S1208 of the first composition may include contacting together the compounds, elements, or components of the first composition. In at least one example embodiment, the preparing S1212 of the second composition may including contacting together the compounds, elements, or components of the second composition. Although both the preparation S1112 of the second composition is illustrated as occurring after the preparation S1208 of the first composition, it should be appreciated that the preparation S1212 of the second composition may occur simultaneously, concurrently, or subsequently with the preparation S1208 of the first composition.

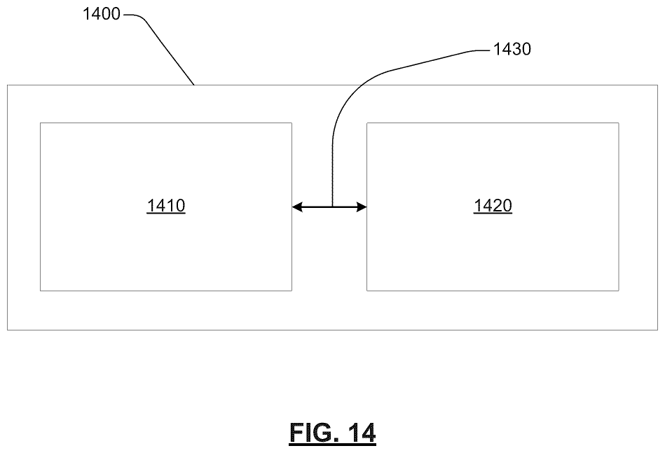

FIG. 13 is a flow diagram illustrating another method for preparing an oral product according to at least one example embodiment.

By way of example, FIG. 13 is a flow diagram illustrating an example method for preparing an oral product (including, for example, the oral products of FIGS. 1-8) according to at least one example embodiment.

As illustrated, in at least one example embodiment, the method includes disposing S1312 a first composition using a three-dimensional printer, disposing S1316 a second composition using the three-dimensional printer, and disposing S1320 a third composition using the three-dimensional printer. As described above, the third composition may be the same as or different from the first composition and/or the second composition, and the second composition may be the same as or different from the first composition. Also as described above, the three-dimensional printer may include a first part or component or portion configured to receive, hold, and dispose the first composition, a second part or component or portion distinct from the first part that is configured to receive, hold, and dispose the second composition, and a third part or component or portion distinct from the first part and/or the second part that is configured to receive, hold, and disposed the third composition. Although the disposing S1316 of the second composition is illustrated as following the disposing S1312 of the first composition, and the disposing S1320 of the third composition is illustrated as following the disposing S1316 of the second composition, it should be appreciated, as described above, that the disposing S1312 of the first composition and/or the disposing S1316 of the second composition and/or the disposing S1320 of the third composition may occur simultaneously, concurrently, or subsequently.

In at least one example embodiment, the method may further include preparing S1300 the first composition and/or preparing S1304 the second composition and/or preparing S1308 the third composition. In at least one example embodiment, the preparing S1300 of the first composition may include contacting together the compounds, elements, or components of the first composition. In at least one example embodiment, the preparing S1304 of the second composition may including contacting together the compounds, elements, or components of the second composition. In at least one example embodiment, the preparing S1308 of the third composition may including contacting together the compounds, elements, or components of the third composition. Although both the preparation S1304 of the second composition is illustrated as occurring after the preparation S1300 of the first composition and the preparation S1308 of the third composition is illustrated as occurring after the preparation S1304 of the second composition, it should be appreciated that the preparation S1308 of the third composition may occur simultaneously, concurrently, or subsequently with the preparation S1300 of the first composition and/or the preparation S1304 of the second composition, and similarly, the preparation S1304 of the second composition may occur simultaneously, concurrently, or subsequently with the preparation S1304 of the second composition.

The present disclosure provides apparatuses, for example, three-dimensional printers, for forming oral products like those detailed above. The apparatuses may each include one or more parts or components or portions configured to receive, hold, and dispose one or more compositions (including, for example, the first composition, the second composition, the third composition, and/or other compositions as detailed above). The apparatus may also include one or more controllers configured to execute one or more programs or acts for one or more of the one or more parts or components or portions.

FIG. 14 is a block diagram illustrating an apparatus for use in preparing an oral product according to at least one example embodiment.

By way of example, FIG. 14 is a block diagram illustrating an example apparatus 1400 for preparing an oral product (including, for example, the oral products of FIGS. 1-8) according to at least one example embodiment.

As illustrated, in at least one example embodiment, the apparatus 1400 includes a part or component or portion 1410 configured to receive or hold or dispose one or more compositions (including, for example, the first composition, the second composition, the third composition, and/or other compositions as detailed above).

In at least one example embodiment, the apparatus 1400 includes a controller 1420 that is configured to execute one or more programs or acts using the part or component or portion 1410. For example, the controller 1420 may be configured to cause the one or more compositions to be disposed (concurrently or subsequently) (including, for example, the printing the first product portion at S912 as illustrated in FIG. 9; the printing the second product portion at S916 as illustrated in FIG. 9; the disposing of the first composition S1208 as illustrated in FIG. 12; the disposing of the second composition S1212 as illustrated in FIG. 1212; the disposing of the first composition S1312 as illustrated in FIG. 13; the disposing of the second composition S1316 as illustrated in FIG. 13; and/or the disposing of the third composition S1320 as illustrated in FIG. 13). As illustrated by line 1430, the controller 1420 may be in electrical communicating with the part or component or portion 1410.

In at least one example embodiment, the controller 1420 may include processing circuitry such as hardware including logic circuits, a hardware/software combination such as a processor executing software, or a combination thereof. The processing circuitry more specifically may include, but is not limited to, a central processing unit (CPU), an arithmetic logic unit (ALU), a digital signal processor, a microcomputer, a field programmable gate array (FPGA), a System-on-Chip (SoC), a programmable logic unit, a microprocessor, application-specific integrated circuit (ASIC), etc.

Although the controller 1420 is illustrated as a single controller it should be appreciated that, in at least one example embodiment, the controller 1420 may include two or more controllers configured independently or together to execute one or more programs or acts using the part or component or portion 1410.

Although the part or component or portion 410 is illustrated as a single part or component or portion it should be appreciated that, in at least one example embodiment, the part or component or portion 1410 may include two or more part or component or portion configured independently or together to receive or hold or dispose the one or more compositions.

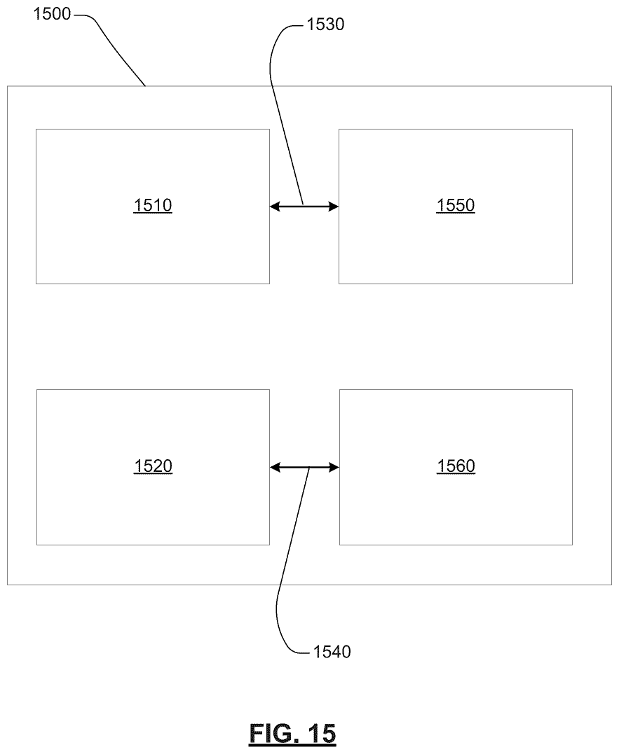

FIG. 15 is a block diagram illustrating another example apparatus for use in preparing an oral product according to at least one example embodiment.

By way of example, FIG. 15 is a block diagram illustrating an example apparatus 500 for preparing an oral product (including, for example, the oral products of FIGS. 1-8) according to at least one example embodiment.

As illustrated, in at least one example embodiment, the apparatus 1500 includes a first part or component or portion 1510 configured to receive or hold or dispose one or more compositions (including, for example, the first composition, the second composition, the third composition, and/or other compositions as detailed above) and a second part or component or portion 520 configured to receive or hold or dispose one or more compositions (including, for example, the first composition, the second composition, the third composition, and/or other compositions as detailed above). In at least one example embodiment, the first part or component or portion 1510 is configured to hold a first composition, while the second part or component or portion 1520 is configured to hold a second composition that is different from the first composition.

In at least one example embodiment, the apparatus 1500 includes a first controller 1550 that is configured to execute one or more programs or acts using the first part or component or portion 1510. For example, the first controller 1550 may be configured to cause the one or more compositions to be disposed (concurrently or subsequently) (including, for example, the printing the first product portion at S912 as illustrated in FIG. 9; the printing the second product portion at S916 as illustrated in FIG. 9; the disposing of the first composition S1208 as illustrated in FIG. 12; the disposing of the second composition S1212 as illustrated in FIG. 1212; the disposing of the first composition S1312 as illustrated in FIG. 13; the disposing of the second composition S1316 as illustrated in FIG. 13; and/or the disposing of the third composition S1320 as illustrated in FIG. 13). In at least one example embodiment, the first controller 1550 may be configured to cause a first composition to be disposed using (or from) the first part or component or portion 1510. As illustrated by line 1530, the controller 1550 may be in electrical communicating with the first part or component or portion 1510.

In at least one example embodiment, the apparatus 1500 includes a second controller 1560 that is configured to execute one or more programs or acts using the second part or component or portion 1520. For example, the second controller 1560 may be configured to cause the one or more compositions to be disposed (concurrently or subsequently) (including, for example, the printing the first product portion at S912 as illustrated in FIG. 9; the printing the second product portion at S916 as illustrated in FIG. 9; the disposing of the first composition S1208 as illustrated in FIG. 12; the disposing of the second composition S1212 as illustrated in FIG. 1212; the disposing of the first composition S1312 as illustrated in FIG. 13; the disposing of the second composition S1316 as illustrated in FIG. 13; and/or the disposing of the third composition S1320 as illustrated in FIG. 13). In at least one example embodiment, the second controller 1560 may be configured to cause a second composition to be disposed using (or from) the second part or component or portion 1520. As illustrated by line 1540, the controller 1550 may be in electrical communicating with the first part or component or portion 1520.

Although not illustrated, it should be appreciated that, in at least one example embodiment, the first controller 1550 may work with the second controller 1560 to execute one or more programs or acts using the second part or component or portion 1520. For example, in at least one example embodiment, the first controller 1550 may be in electrical communication with the second controller 1560 and/or the second part or component or portion 1520.

Although not illustrated, it should be appreciated that, in at least one example embodiment, the second controller 1560 may work with the first controller 1550 to execute one or more programs or acts using the first part or component or portion 1510. For example, in at least one example embodiment, the second controller 1560 may be in electrical communication with the first controller 1550 and/or the first part or component or portion 1510.

In at least one example embodiment, the first controller 1550 may include processing circuitry such as hardware including logic circuits, a hardware/software combination such as a processor executing software, or a combination thereof. The processing circuitry more specifically may include, but is not limited to, a central processing unit (CPU), an arithmetic logic unit (ALU), a digital signal processor, a microcomputer, a field programmable gate array (FPGA), a System-on-Chip (SoC), a programmable logic unit, a microprocessor, application-specific integrated circuit (ASIC), etc.

In at least one example embodiment, the second controller 1560 may include processing circuitry such as hardware including logic circuits, a hardware/software combination such as a processor executing software, or a combination thereof. The processing circuitry more specifically may include, but is not limited to, a central processing unit (CPU), an arithmetic logic unit (ALU), a digital signal processor, a microcomputer, a field programmable gate array (FPGA), a System-on-Chip (SoC), a programmable logic unit, a microprocessor, application-specific integrated circuit (ASIC), etc.

Although only the first controller 1550 is illustrated as a single component it should be appreciated that, in at least one example embodiment, the first controller 1550 may include two or more controllers configured independently or together to execute (alone or with the second controller 1560) one or more programs or acts using the first part or component or portion 1510 and/or the second part or component or portion 1520.

Although only the second controller 1560 is illustrated as a single component it should be appreciated that, in at least one example embodiment, the second controller 1560 may include two or more controllers configured independently or together to execute (alone or with the first controller 1550) one or more programs or acts using the first part or component or portion 1510 and/or the second part or component or portion 1520.

Although the first part or component or portion 1510 is illustrated as a single part or component or portion it should be appreciated that, in at least one example embodiment, the first part or component or portion 1510 may include two or more part or component or portion configured independently or together to receive or hold or dispose the one or more compositions.

Although the second part or component or portion 1520 is illustrated as a single part or component or portion it should be appreciated that, in at least one example embodiment, the second part or component or portion 1520 may include two or more part or component or portion configured independently or together to receive or hold or dispose the one or more compositions.

While some example embodiments have been disclosed herein, it should be understood that other variations may be possible. Such variations are not to be regarded as a departure from the spirit and scope of the present disclosure, and all such modifications as would be obvious to one skilled in the art are intended to be included within the scope of the following claims.

Although described with reference to specific examples and drawings, modifications, additions, and substitutions of example embodiments may be variously made according to the description by those of ordinary skill in the art. For example, the described techniques may be performed in an order different with that of the methods described, and/or elements such as the described system, architecture, devices, circuit, and the like, may be connected or combined to be different from the above-described methods, or results may be appropriately achieved by other elements or equivalents.

What is claimed is:

1. A method of making an oral product, the method comprising:
printing a first product portion of an oral product using a three-dimensional printer, the first product portion having a first composition; and
printing a second product portion of the oral product using the three-dimensional printer, the second product portion having a second composition different from the first composition, the first product portion and the second product portion defining distinct, non-mixed regions of the oral product.

2. The method of claim 1, wherein
the printing the second product portion includes printing the second product portion directly on the first product portion.

3. The method of claim 1, wherein
the printing the first product portion includes discharging the first composition from the three-dimensional printer as a liquid and cooling the first composition such that the first product portion is a solid, and
the printing the second product portion includes discharging the second composition from the three-dimensional printer as a liquid and cooling the second composition such that the second product portion is a solid.

4. The method of claim 3, wherein the cooling the first composition includes circulating air in a region surrounding the first composition.

5. The method of claim 3, wherein the printing the first product portion is completed prior to beginning the printing the second product portion.

6. The method of claim 1, further comprising:
preparing the first composition prior to printing the first product portion, and
preparing the second composition prior to printing the second product portion.

7. The method of claim 6, wherein the preparing the first composition includes heating the first composition to a first temperature.

8. The method of claim 7, wherein the first temperature ranges from 150° C. to 160° C.

9. The method of claim 8, wherein the preparing the second composition includes heating the second composition to a second temperature ranging from 150° C. to 160° C.

10. The method of claim 6, further comprising:
preheating the three-dimensional printer; and
after the preheating, transferring the first composition from a first vessel to the three-dimensional printer.

11. The method of claim 10, further comprising:
preheating the three-dimensional printer; and
after the preheating the three-dimensional printer, transferring the second composition from a second vessel to the three-dimensional printer.

12. The method of claim 1, wherein
the printing the first product portion is performed using a first part of the three-dimensional printer; and
the printing the second product portion is performed using a second part of the three-dimensional printer.

13. The method of claim 12, wherein the printing the first product portion includes,
heating a first region of the first part to a first temperature, and
heating a second region of the first part to a second temperature, the second region downstream of the first region.

14. The method of claim 13, wherein the second temperature is different than the first temperature.

15. The method of claim 14, wherein the second temperature is higher than the first temperature.

16. The method of claim 15, wherein
the first temperature ranges from 80° C. to 85° C., and
the second temperature ranges from 90° C. to 105° C.

17. The method of claim 13, wherein
the first region is a first interior region at least partially defined by a syringe, and
the second region is a second interior region at least partially defined by a nozzle coupled to the syringe.

18. The method of claim 1, wherein the printing the first product portion includes printing a first layer having a first thickness of less than 1 mm.

19. The method of claim 18, wherein the printing the second product portion includes printing a second layer having a second thickness of less than 1 mm.

20. The method of claim 1, wherein
the printing the first product portion includes printing a plurality of first product portions, and
the printing the second product portion includes printing a plurality of second product portions.

21. The method of claim 1, further comprising:
printing a third product portion of the oral product using the three-dimensional printer, the third product portion having a third composition different from the first composition and the second composition, the first product portion, the second product portion, and the third product portion defining distinct, non-mixed regions of the oral product.

22. The method of claim 1, further comprising:
controlling a humidity to a desired humidity during the printing the first product portion and the printing the second product portion.

23. The method of claim 22, wherein the desired humidity ranges from 40% to 60%.

24. A method for preparing an oral product, the method comprising:
disposing a first composition using a first part of a three-dimensional printer to form one or more dissolvable first portions of the oral product; and
disposing a second composition different from the first composition using a second part of the three-dimensional printer to form one or more dissolvable second portions of the oral product, at least one of the first composition and the second composition including nicotine.

25. An oral product comprising:
a first portion having a first composition and defining a first thickness less than 1 mm, the first portion being homogeneous; and
a second portion having a second composition and defining a second thickness less than 1 mm, the second portion being homogeneous,
the second portion directly adjacent to the first portion, and
the first composition non-mixed with the second composition.

26. The oral product of claim 25, wherein at least one of the first composition and the second composition includes nicotine.

27. The oral product of claim 26, wherein the other of the first composition and the second composition includes a nicotine masking agent.

28. The oral product of claim 26, wherein
the first composition includes a first amount of nicotine, and
the second composition includes a second amount of nicotine different than the first amount of nicotine.

29. The oral product of claim 25, wherein
the first composition has a first density, and
the second composition has a second density different than the first density.

30. The oral product of claim 25, wherein
the first portion is a first layer, and
the second portion is a second layer.

31. The oral product of claim 25, wherein the oral product is a lozenge.

32. The oral product of claim 25, wherein the first portion and the second portion are preparing using three-dimensional printing.
